(19) United States
(12) Patent Application Publication
(10) Pub. No.: US 2006/0036080 A1
(43) Pub. Date: Feb. 16, 2006

(54) Conjugates of GM-CSF moiety and a polymer

(76) Inventors: Mary J. Bossard, Madison, AL (US); Ping Zhang, Madison, AL (US)

Correspondence Address: NEKTAR THERAPEUTICS, 150 INDUSTRIAL ROAD, SAN CARLOS, CA, 94070, US

(21) Appl. No.: 11/181,692

(22) Filed: Jul. 14, 2005

Related U.S. Application Data
(60) Provisional application No. 60/588,601, filed on Jul. 16, 2004.

(51) Int. Cl. C08G 63/91; C07K 14/535

(52) U.S. Cl. 530/351; 525/54.1

(57) ABSTRACT

Conjugates of a GM-CSF moiety and one or more water-soluble polymers are provided. Typically, the water-soluble polymer is poly(ethylene glycol) or a derivative thereof. Also provided are compositions comprising conjugates, methods of making conjugates, and methods of administering compositions comprising conjugates to a patient.

### CROSS REFERENCE TO RELATED APPLICATION

This application claims the benefit of priority to U.S. Provisional Patent Application Ser. No. 60/588,601, filed Jul. 16, 2004, which is incorporated herein by reference.

### FIELD OF THE INVENTION

The present invention relates generally to conjugates comprising a GM-CSF moiety (i.e., a moiety having GM-CSF activity) and a polymer. In addition, the invention relates to (among other things) compositions comprising the conjugates, methods for synthesizing the conjugates, and methods for delivering the conjugates.

### BACKGROUND OF THE INVENTION

One important function of the human hematopoietic system is the replacement of a variety of white blood cells (including macrophages, neutrophils, and basophils/mast cells), red blood cells (i.e., erythrocytes) and clot-forming cells (e.g., megakaryocytes/platelets). Each of these specialized cells is formed from hematopoietic precursor cells located in the bone marrow. Specific hormone-like glycoproteins called “colony stimulating factors” control the differentiation and maturation of the hematopoietic precursor cells into any one of the number of specialized blood cells.

One such colony stimulating factor is granulocyte macrophage-colony stimulating factor or “GM-CSF.” As its name implies, this colony stimulating factor promotes the proliferation and differentiation of white blood cells such as granulocytes and macrophages, although GM-CSF can promote the formation of other cell types as well. GM-CSF is produced by a number of different cell types (including activated T cells, B cells, macrophages, mast cells, endothelial cells and fibroblasts) in response to cytokine, immune and inflammatory stimuli. Native GM-CSF is a glycoprotein of 127 amino acids and can have a variety of molecular weights depending on the extent of glycosylation.

Pharmacologically, GM-CSF has been administered to cancer patients in order to accelerate the replacement of white blood cells that are killed during chemotherapy treatments. With a similar aim to accelerate white blood cell replacement, this colony stimulating factor has been administered to leukemia patients undergoing bone marrow replacement therapy. Additional applications, such as accelerated wound healing, have been proposed. See, for example, U.S. Pat. No. 6,689,351.

One drawback associated with current forms of GM-CSF therapy is the frequency of dosing. Because GM-CSF therapy typically requires daily injections, patients dislike the inconvenience and discomfort associated with this regimen. Coupled with the fact that patients require frequent blood testing to determine white blood cells counts (which require trips to a health care practitioner), many patients would prefer an alternative that is less cumbersome and/or involves a reduction in the number of injections.

One proposed solution to these problems has been to provide a prolonged release form of GM-CSF. For example, U.S. Pat. No. 5,942,253 describes microspheres of poly(lactic acid-co-glycolic acid) or other biodegradable polymers of GM-CSF. The formation of microspheres, however, can be a complex process, requiring several synthetic steps. Thus, this prolonged release approach suffers from complexities that are ideally avoided.

PEGylation, or the attachment of a poly(ethylene glycol) derivative to a protein, has been described as a means to prolong a protein's in vivo half-life, thereby resulting in prolonged pharmacologic activity. For example, U.S. Pat. No. 5,880,255 describes a conjugate of GM-CSF and poly(ethylene glycol) formed from a reaction with 2,2,2-trifluoroethanesulfonate derivatized linear monomethoxy poly(ethylene glycol) having a molecular weight of 5,000 Daltons. Notwithstanding this described conjugate however, there remains a need for other conjugates of GM-CSF possessing, for example, a polymer having a molecular weight greater than 5,000 Daltons, a polymer having a different structure (e.g., a branched and/or forked structure), different attachment sites, site-specific or site-selective attachment sites, and so forth.

Thus, there remains a need in the art to provide additional GM-CSF moiety-polymer conjugates. Among other things, one or more embodiments of the present invention is therefore directed to such conjugates as well as compositions comprising the conjugates and related methods as described herein, which are believed to be new and completely unsuggested by the art.

### SUMMARY OF THE INVENTION

Accordingly, in one or more embodiments of the invention, a conjugate is provided, the conjugate comprising the following structure: 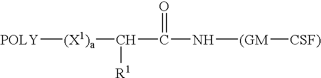
wherein:
POLY is a water-soluble polymer;
(a) is either zero or one;
X¹, when present, is a spacer moiety comprised of one or more atoms;
R¹ is an organic radical;
GM-CSF is a GM-CSF moiety.

In one or more embodiments of the invention, a conjugate is provided, the conjugate comprising the following structure: 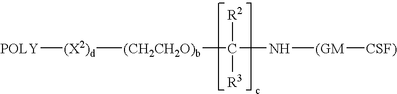
wherein:
POLY is a water-soluble polymer;
X is a spacer moiety comprised of one or more atoms;
(b) is zero or an integer having a value of one through 10 (i.e., 1, 2, 3, 4, 5, 6, 7, 8, 9 or 10);
(c) is zero or an integer having a value of one through 10 (i.e., 1, 2, 3, 4, 5, 6, 7, 8, 9 or 10);
R², in each occurrence, is independently H or an organic radical;
R³, in each occurrence, is independently H or an organic radical; and
GM-CSF is a GM-CSF moiety.

In one or more embodiments of the invention, a conjugate is provided, the conjugate comprising a GM-CSF moiety comprising an internal amine covalently attached, either directly or through a spacer moiety comprised of one or more atoms, to a branched water-soluble polymer.

In one or more embodiments of the invention, a pharmaceutical composition is provided, the composition comprising a conjugate as provided herein.

In one or more embodiments of the invention, a pharmaceutical composition is provided, the composition comprising (i) a conjugate comprising a human GM-CSF covalently attached, either directly or through a spacer moiety comprised of one or more atoms, to a water-soluble polymer, wherein the water-soluble polymer has a weight-average molecular weight of greater than 5,000 Daltons, with the proviso that when the water-soluble polymer is a branched water-soluble polymer, the branched water-soluble polymer does not include a lysine residue; and (ii) a pharmaceutically acceptable excipient, wherein at least about 85% of the conjugates in the composition will have a total of from one to two polymers attached to the human GM-CSF.

In one or more embodiments of the invention, a method for delivering a conjugate to a patient is provided, the method comprising the step of administering to the patient a pharmaceutical composition as provided herein.

In one or more embodiments of the invention, a method for making a conjugate is provided, the method comprising contacting, under conjugation conditions, a GM-CSF moiety with a polymeric reagent to result in a conjugate and/or composition as provided herein.

### BRIEF DESCRIPTION OF THE FIGURES

FIG. 1 is the chromatogram following SEC-HPLC analysis of a conjugate solution as described in Example 1

FIG. 2 is the chromatogram following anion-exchange purification of a composition described in Example 1.

FIG. 3 shows the SDS-PAGE results of conjugate fractions as described in Example 1.

FIG. 4 is the chromatogram following SEC-HPLC analysis of a conjugate solution as described in Example 2.

FIG. 5 is the chromatogram following anion-exchange purification of a composition as described in Example 3.

FIG. 6 is the chromatogram following SEC-HPLC analysis of a conjugate solution as described in Example 4.

FIG. 7 is the chromatogram following SEC-HPLC analysis of a conjugate solution as described in Example 5.

FIG. 8 is the chromatogram following SEC-HPLC analysis of a conjugate solution as described in Example 6.

### DETAILED DESCRIPTION OF THE INVENTION

Before describing one or more embodiments of the present invention in detail, it is to be understood that this invention is not limited to the particular polymers, synthetic techniques, GM-CSF moieties, and the like, as such may vary.

It must be noted that, as used in this specification and the claims, the singular forms “a,” “an,” and “the” include plural referents unless the context clearly dictates otherwise. Thus, for example, reference to “a polymer” includes a single polymer as well as two or more of the same or different polymers, reference to “a pharmaceutically acceptable excipient” refers to a single pharmaceutically acceptable excipient as well as two or more of the same or different pharmaceutically acceptable excipients, and the like.

In describing and claiming the present invention(s), the following terminology will be used in accordance with the definitions provided below.

“PEG,” “polyethylene glycol” and “poly(ethylene glycol)” as used herein, are interchangeable. Typically, PEGs for use in accordance with the invention comprise the following structure: “—(OCH₂CH₂)ₙ—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. Throughout the specification and claims, it should be remembered that the term “PEG” includes structures having various terminal or “end capping” groups. The term “PEG” also means a polymer that contains a majority, that is to say, greater than 50%, of —OCH₂CH₂— or —CH₂CH₂O-repeating subunits. With respect to specific forms, the PEG can take any number of a variety of molecular weights, as well as structures or geometries such as “branched,” “linear,” “forked,” “multifunctional,” and the like, to be described in greater detail below.

The terms “end-capped” and “terminally capped” are interchangeably used herein to refer to a terminal or endpoint of a polymer having an end-capping moiety. Typically, although not necessarily, the end-capping moiety comprises a hydroxy or C₁₋₂₀ alkoxy group, more preferably a C₁₋₁₀ alkoxy group, and still more preferably a C₁₋₅ alkoxy group. Thus, examples of end-capping moieties include alkoxy (e.g., methoxy, ethoxy and benzyloxy), as well as aryl, heteroaryl, cyclo, heterocyclo, and the like. It must be remembered that the end-capping moiety may include one or more atoms of the terminal monomer in the polymer [e.g., the end-capping moiety “methoxy” in CH₃(OCH₂CH₂)ₙ—]. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount and/or location of the polymer and/or the moiety (e.g., active agent) to which the polymer is coupled can be determined by using a suitable detector. Such labels include, without limitation, fluorescers, chemiluminescers, moieties used in enzyme labeling, colorimetric moieties (e.g., dyes), metal ions, radioactive moieties, and the like. Suitable detectors include photometers, films, spectrometers, and the like. The end-capping group can also advantageously comprise a phospholipid. When the polymer has an end-capping group comprising a phospholipid, unique properties are imparted to the polymer and the resulting conjugate. Exemplary phospholipids include, without limitation, those selected from the class of phospholipids called phosphatidylcholines. Specific phospholipids include, without limitation, those selected from the group consisting of dilauroylphosphatidylcholine, dioleylphosphatidylcholine, dipalmitoylphosphatidylcholine, disteroylphosphatidylcholine, behenoylphosphatidylcholine, arachidoylphosphatidylcholine, and lecithin.

“Non-naturally occurring” with respect to a polymer as described herein, means a polymer that in its entirety is not found in nature. A non-naturally occurring polymer may, however, contain one or more monomers or segments of monomers that are naturally occurring, so long as the overall polymer structure is not found in nature.

The term “water soluble” as in a “water-soluble polymer” is any polymer that is soluble in water at room temperature. Typically, a water-soluble polymer will transmit at least about 75%, more preferably at least about 95%, of light transmitted by the same solution after filtering. On a weight basis, a water-soluble polymer will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer is about 95% (by weight) soluble in water or completely soluble in water.

Molecular weight in the context of a water-soluble polymer, such as PEG, can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Other methods for measuring molecular weight values can also be used, such as the use of end-group analysis or the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03. As used herein, references will at times be made to a single water-soluble polymer having either a weight-average molecular weight or number-average molecular weight; such references will be understood to mean that the single-water soluble polymer was obtained from a composition of water-soluble polymers having the stated molecular weight.

The terms “active” or “activated” when used in conjunction with a particular functional group, refer to a reactive functional group that reacts readily with an electrophile or a nucleophile on another molecule. This is in contrast to those groups that require strong catalysts or highly impractical reaction conditions in order to react (i.e., a “non-reactive” or “inert” group).

As used herein, the term “functional group” or any synonym thereof is meant to encompass protected forms thereof as well as unprotected forms.

The terms “spacer moiety,” “linkage” or “linker” are used herein to refer to an atom or a collection of atoms used to link interconnecting moieties such as a terminus of a polymer and a GM-CSF moiety or an electrophile or nucleophile of a GM-CSF moiety. The spacer moiety may be hydrolytically stable or may include a physiologically hydrolyzable or enzymatically degradable linkage.

“Alkyl” refers to a hydrocarbon chain, typically ranging from about 1 to 15 atoms in length. Such hydrocarbon chains are preferably but not necessarily saturated and may be branched or straight chain, although typically straight chain is preferred. Exemplary alkyl groups include methyl, ethyl, propyl, butyl, pentyl, 1-methylbutyl, 1-ethylpropyl, 3-methylpentyl, and the like. As used herein, “alkyl” includes cycloalkyl as well as cycloalkylene-containing alkyl.

“Lower alkyl” refers to an alkyl group containing from 1 to 6 carbon atoms, and may be straight chain or branched. Nonlimiting examples of lower alkyl include methyl, ethyl, n-butyl, i-butyl, and t-butyl.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8 carbon atoms. “Cycloalkylene” refers to a cycloalkyl group that is inserted into an alkyl chain by bonding of the chain at any two carbons in the cyclic ring system.

“Alkoxy” refers to an —O—R group, wherein R is alkyl or substituted alkyl, preferably C₁₋₆ alkyl (e.g., methoxy, ethoxy, propyloxy, and so forth).

The term “substituted” as in, for example, “substituted alkyl,” refers to a moiety (e.g., an alkyl group) substituted with one or more noninterfering substituents, such as, but not limited to: alkyl, C₃₋₈ cycloalkyl, e.g., cyclopropyl, cyclobutyl, and the like; halo, e.g., fluoro, chloro, bromo, and iodo; cyano; alkoxy; lower phenyl; substituted phenyl; and the like. “Substituted aryl” is aryl having one or more noninterfering substituents. For substitutions on a phenyl ring, the substituents may be in any orientation (i.e., ortho, meta, or para).

“Noninterfering substituents” are those groups that, when present in a molecule, are typically nonreactive with other functional groups contained within the molecule.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl and heterocycle.

“Heteroaryl” is an aryl group containing from one to four heteroatoms, preferably sulfur, oxygen, or nitrogen, or a combination thereof. Heteroaryl rings may also be fused with one or more cyclic hydrocarbon, heterocyclic, aryl, or heteroaryl rings. As used herein, “heteroaryl” includes substituted heteroaryl.

“Heterocycle” or “heterocyclic” means one or more rings of 5-12 atoms, preferably 5-7 atoms, with or without unsaturation or aromatic character and having at least one ring atom that is not a carbon. Preferred heteroatoms include sulfur, oxygen, and nitrogen. As used herein, “heterocycle” includes substituted heterocycle.

“Substituted heteroaryl” is heteroaryl having one or more noninterfering groups as substituents.

“Substituted heterocycle” is a heterocycle having one or more side chains formed from noninterfering substituents.

An “organic radical” as used herein shall include alkyl, substituted alkyl, aryl and substituted aryl.

“Electrophile” and “electrophilic group” refer to an ion or atom or collection of atoms, that may be ionic, having an electrophilic center, i.e., a center that is electron seeking, capable of reacting with a nucleophile.

“Nucleophile” and “nucelophilic group” refer to an ion or atom or collection of atoms that may be ionic having a nucleophilic center, i.e., a center that is seeking an electrophilic center or capable of reacting with an electrophile.

A “hydrolytically degradable” or “hydrolyzable” linkage or bond is a bond that reacts with water (i.e., is hydrolyzed) under physiological conditions. Preferred are bonds that have a hydrolysis half-life at pH 8, 25° C. of less than about 30 minutes. The tendency of a bond to hydrolyze in water will depend not only on the general type of linkage connecting two given atoms but also on the substituents attached to the two given atoms. Appropriate hydrolytically unstable or weak linkages include but are not limited to carboxylate ester, phosphate ester, anhydrides, acetals, ketals, acyloxyalkyl ether, imine, orthoester, peptide and oligonucleotide.

An “enzymatically degradable linkage” means a linkage that is subject to degradation by one or more enzymes.

A “hydrolytically stable” linkage or bond refers to a chemical bond, typically a covalent bond, that is substantially stable in water, that is to say, does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethane, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

“Pharmaceutically acceptable excipient” refers to an excipient that may optionally be included in a composition and that causes no significant adverse toxicological effects to a patient upon administration.

“Therapeutically effective amount” is used herein to mean the amount of a polymer-(GM-CSF) moiety conjugate that is needed to provide a desired level of the conjugate (or corresponding unconjugated GM-CSF moiety) in the bloodstream or in the target tissue. The precise amount will depend upon numerous factors, e.g., the particular GM-CSF moiety, the components and physical characteristics of the therapeutic composition, the intended patient population, the mode of delivery, individual patient considerations, and the like, and can readily be determined by one skilled in the art.

“Multi-functional” means a polymer having three or more functional groups contained therein, where the functional groups may be the same or different. Multi-functional polymeric reagents will typically contain from about 3-100 functional groups, or from 3-50 functional groups, or from 3-25 functional groups, or from 3-15 functional groups, or from 3 to 10 functional groups, or will contain 3, 4, 5, 6, 7, 8, 9 or 10 functional groups within the polymer backbone.

The term “GM-CSF moiety,” as used herein, refers to a moiety having GM-CSF activity. The GM-CSF moiety will also have at least one electrophilic group or nucleophilic group suited for reaction with a polymeric reagent. The GM-CSF moiety is a protein, i.e., comprised of a series of monomers made of amino acid, optionally glycosylated in one or more locations. In addition, the term “GM-CSF moiety” encompasses both the GM-CSF moiety prior to conjugation as well as the GM-CSF moiety residue following conjugation. As will be explained in further detail below, one of ordinary skill in the art can determine whether any given moiety has GM-CSF activity. A protein comprising an amino acid sequence corresponding to the sequences selected from the group consisting of SEQ ID NO: 1, SEQ ID NO: 2 and SEQ ID NO: 3, is a GM-CSF moiety, as well as any protein or polypeptide substantially homologous thereto and any of SEQ ID NOs 1, 2 and 3 beginning with a methionyl residue, whose biological properties result in the activity of GM-CSF in both instances. As used herein, the term “GM-CSF moiety” includes proteins modified deliberately, as for example, by site directed mutagenesis or accidentally through mutations. The term “GM-CSF moiety” also includes derivatives having from 1 to 6 additional glycosylation sites, derivatives having at least one additional amino acid at the carboxy terminal end of the protein wherein the additional amino acid(s) includes at least one glycosylation site, and derivatives having an amino acid sequence which includes at least one glycosylation site.

The term “substantially homologous” means that a particular subject sequence, for example, a mutant sequence, varies from a reference sequence by one or more substitutions, deletions, or additions, the net effect of which does not result in an adverse functional dissimilarity between the reference and subject sequences. For purposes of the present invention, sequences having greater than 95 percent homology, equivalent biological properties (although potentiality different degrees of activity), and equivalent expression characteristics are considered substantially homologous. For purposes of determining homology, truncation of the mature sequence should be disregarded. Sequences having lesser degrees of homology, comparable bioactivity, and equivalent expression characteristics are considered substantial equivalents. Exemplary GM-CSF moieties for use herein include those proteins having a sequence that is substantially homologous to one or more of the sequences selected from the group consisting of SEQ ID NO: 1, SEQ ID NO: 2 and SEQ ID NO: 3.

The term “fragment” means any protein or polypeptide having the amino acid sequence of a portion of a GM-CSF moiety that retains some degree of GM-CSF activity. Fragments include proteins or polypeptides produced by proteolytic degradation of the GM-CSF protein or produced by chemical synthesis by methods routine in the art. Determining whether a particular fragment has GM-CSF activity can be carried out by one of ordinary skill. An appropriate test which can be utilized to demonstrate such activity is described herein.

A “deletion variant” of a GM-CSF moiety is peptide or protein in which one amino acid residue of the GM-CSF moiety has been deleted and the amino acid residues preceding and following the deleted amino acid residue are connected via an amide bond (except in instances where the deleted amino acid residue was located on a terminus of the peptide or protein). Deletion variants include instances where only a single amino acid residue has been deleted, as well as instances where two amino acids are deleted, three amino acids are deleted, four amino acids are deleted, and so forth. Each deletion variant must, however, retain some degree of GM-CSF activity.

A “substitution variant” of a GM-CSF moiety is peptide or protein in which one amino acid residue of the GM-CSF moiety has been deleted and a different amino acid residue has taken its place. Substitution variants include instances where only a single amino acid residue has been substituted, as well as instances where two amino acids are substituted, three amino acids are substituted, four amino acids are substituted, and so forth. Each substitution variant must, however, have some degree of GM-CSF activity.

An “addition variant” of a GM-CSF moiety is peptide or protein in which one amino acid residue of the GM-CSF has been added into an amino acid sequence and adjacent amino acid residues are attached to the added amino acid residue by way of amide bonds (except in instances where the added amino acid residue is located on a terminus of the peptide or protein, wherein only a single amide bond attaches the added amino acid residue). Addition variants include instances where only a single amino acid residue has been added, as well as instances where two amino acids are added, three amino acids are added, four amino acids are added, and so forth. Each addition variant must, however, have some degree of GM-CSF activity.

The term “patient,” refers to a living organism suffering from or prone to a condition that can be prevented or treated by administration of an active agent (e.g., conjugate), and includes both humans and animals.

“Optional” or “optionally” means that the subsequently described circumstance may or may not occur, so that the description includes instances where the circumstance occurs and instances where it does not.

“Substantially” (unless specifically defined for a particular context elsewhere or the context clearly dictates otherwise) means nearly totally or completely, for instance, satisfying one or more of the following: greater than 50%, 51% or greater, 75% or greater, 80% or greater, 90% or greater, and 95% or greater of the condition.

Unless the context clearly dictates otherwise, when the term “about” precedes a numerical value, the numerical value is understood to mean ±10% of the stated numerical value.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutarnine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

Turning to one or more embodiments of the invention, a conjugate is provided, the conjugate comprising a GM-CSF moiety covalently attached, either directly or through a spacer moiety comprised of one or more atoms, to a water-soluble polymer. The conjugates of the invention will have one or more of the following features.

The GM-CSF Moiety

As previously stated, the term “GM-CSF moiety” shall include the GM-CSF moiety prior to conjugation as well as to the GM-CSF moiety following attachment (either directly or through a spacer moiety) to a water-soluble polymer. It will be understood, however, that when the GM-CSM moiety is attached (either directly or through a spacer moiety) to a water-soluble polymer, the GM-CSF moiety is slightly altered due to the presence of one or more covalent bonds associated with linkage to the polymer (or spacer moiety that is attached to the polymer). Often, this slightly altered form of the GM-CSF moiety attached to another molecule is referred to a “residue” of the GM-CSF moiety.

The GM-CSF moiety can be derived from either non-recombinant methods or from recombinant methods and the invention is not limited in this regard.

The GM-CSF moiety can be derived non-recombinantly. For example, the GM-CSF can be obtained from blood-derived sources. In particular, GM-CSF can be isolated from human plasma or tissues using techniques (e.g., precipitation techniques, centrifugation techniques, chromatographic techniques) known to those of ordinary skill in the art.

The GM-CSF moiety can be derived from recombinant methods. For example, the cDNA coding for human GM-CSF (a preferred GM-CSF moiety) has been isolated, characterized, and cloned into expression vectors. See, e.g., U.S. Pat. Nos. 5,078,996 and 5,891,429, and Wong et al. (1985) “Human GM-CSF: Molecular Cloning of the Complementary DNA and Purification of the Natural and Recombinant Proteins,” Science 218:819, and Cantrell et al. (1985) “Cloning, Sequence, and Expression of a Human Granulocyte/Macrophage Colony-Stimulating Factor,” Proc. Natl. Acad. Sci. U.S.A., Vol. 82: 6250. GM-CSF moieties expressed in bacterial (Escherichia coli), mammalian (e.g., Chinese hamster ovary cells), and yeast (e.g., Saccharomyces cerevisiae) expression systems can be used.

Once expressed, endogenous human GM-CSF is a monomeric glycoprotein with a molecular weight of about 22,000 Daltons. The expressed amino acid sequence is provided as SEQ ID NO: 1. Preferred for use as a GM-CSF moiety herein are any of a number amino acid sequences of human GM-CSF. At least three different human GM-CSF proteins have been produced in various expression systems: sargramostim; molgramostim; regramostim; and ecogramostim. Sargramostim is expressed in Saccharomyces cerevisiae, has an amino acid substitution of leucine at position 23 (SEQ ID NO: 2) as compared to endogenous human GM-CSF and is O-glycosylated. Molgramostim is expressed in Escherichia coli and is nonglycosylated. Regramostim is produced in hamster ovary cells (CHO) cells and is fully glycosylated. Methionyl versions of these proteins are also contemplated wherein a methione residue precedes the complete amino acid sequence. Unless specifically noted, all assignments of a numeric location of an amino acid residue as provided herein are based on SEQ ID NO: 1.

Exemplary recombinant methods used to prepare a GM-CSF moiety (whether a human GM-CSF or a different protein having GM-CSF activity) can be briefly described. Such methods involve constructing the nucleic acid encoding the desired polypeptide or fragment, cloning the nucleic acid into an expression vector, transforming a host cell (e.g., plant, bacteria such as Escherichia coli, yeast such as Saccharomyces cerevisiae, or mammalian cell such as Chinese hamster ovary cell or baby hamster kidney cell), and expressing the nucleic acid to produce the desired polypeptide or fragment. The expression can occur via exogenous expression (when the host cell naturally contains the desired genetic coding) or via endogenous expression. Methods for producing and expressing recombinant polypeptides in vitro and in prokaryotic and eukaryotic host cells are known to those of ordinary skill in the art. See, for example, U.S. Pat. No. 4,868,122.

To facilitate identification and purification of the recombinant polypeptide, nucleic acid sequences that encode for an epitope tag or other affinity binding sequence can be inserted or added in-frame with the coding sequence, thereby producing a fusion protein comprised of the desired polypeptide and a polypeptide suited for binding. Fusion proteins can be identified and purified by first running a mixture containing the fusion protein through an affinity column bearing binding moieties (e.g., antibodies) directed against the epitope tag or other binding sequence in the fusion proteins, thereby binding the fusion protein within the column. Thereafter, the fusion protein can be recovered by washing the column with the appropriate solution (e.g., acid) to release the bound fusion protein. The recombinant polypeptide can also be identified and purified by lysing the host cells, separating the polypeptide, e.g., by size exclusion chromatography, and collecting the polypeptide. These and other methods for identifying and purifying recombinant polypeptides are known to those of ordinary skill in the art. In one or more embodiments of the present invention, however, it is preferred that the GM-CSF moiety is not in the form of a fusion protein.

Depending on the system used to express proteins having GM-CSF activity, the GM-CSF moiety can be unglycosylated or glycosylated and either may be used. That is, the GM-CSF moiety can be unglycosylated or the GM-CSF moiety can be glycosylated. In one or more embodiments of the invention, it is preferred that the GM-CSF moiety is glycosylated. Examples of glycosylation include O-glycosylation and N-glycosylation. It is believed that the glycosylation sites of endogenous human GM-CSF are serine 9 (O-glycosylation), threonine 10 (O-glycosylation), asparagine 27 (N-glycosylation) and asparagine 37 (N-glycosylation). Preferred glycosylation arrangements of any GM-CSF moiety will occur at these sites (or sites corresponding to these locations on the given GM-CSF moiety). Thus, the GM-CSF moiety can have a degree of glycosylation selected from the group consisting of: no glycosylation, glycosylation at a single site; glycosylation at two sites; glycosylation at three sites; and glycosylation at four sites. A particularly preferred glycosylation arrangement is O-glycosylation only at serine 9 and threonine 10 and without N-glycosylation.

The moiety having GM-CSF activity can advantageously be modified to include one or more amino acid residues such as, for example, lysine, cysteine and/or arginine, in order to provide facile attachment of a polymer to an atom within an amino acid. For example, U.S. Pat. No. 6,608,183 describes “cysteine-added” sequences of GM-CSF that can be used as a GM-CSF moiety and methods for preparing such “cysteine-added” sequences. In addition, the GM-CSF moiety can be modified to include a non-naturally occurring amino acid residue. Techniques for adding amino acid residues and non-naturally occurring amino acid residues are well known to those of ordinary skill in the art. See, for example, U.S. Pat. No. 5,393,870 and J. March, Advanced Organic Chemistry: Reactions Mechanisms and Structure, 4th Ed. (New York: Wiley-Interscience, 1992).

In addition, the GM-CSF moiety can advantageously be modified to include attachment of a functional group (other than through addition of a functional group-containing amino acid residue). For example, the GM-CSF moiety can be modified to include a thiol group. In addition, the GM-CSF moiety can be modified to include an N-terminal alpha carbon. In addition, the GM-CSF moiety can be modified to include one or more carbohydrate moieties. GM-CSF moieties modified to contain an aminoxy, aldehyde or other functional group can also be used. Furthermore, oxidized variants of GM-CSF can be used as a GM-CSF moiety. See, for example, U.S. Pat. No. 5,358,707. Derivatives of GM-CSF are also included as GM-CSF moieties. See U.S. Pat. No. 5,298,603.

Nonlimiting examples of GM-CSF moieties include the following: a human GM-CSF; hybrid proteins having GM-CSF activity, and peptide mimetics having GM-CSF activity. Biologically active fragments, deletion variants, substitution variants or addition variants of any of the foregoing that maintain at least some degree of GM-CSF activity can also serve as a GM-CSF moiety.

For any given moiety, it is possible to determine whether that moiety has GM-CSF activity. For example, as described in U.S. Pat. No. 5,393,870, human bone marrow from the iliac crest of healthy donors can be collected, placed into solution and centrifuged, with cells being collected and diluted for subsequent culturing. Following culturing, each colony of cells can be identified and the proposed GM-CSF moiety can be added to the appropriate colony and tested for accelerated proliferation relative to a control. Other methods known to those of ordinary skill in the art can also be used to determine whether a given moiety has GM-CSF activity. Such methods are useful for determining the GM-CSF activity of both the moiety itself (and therefore can be used as a “GM-CSF moiety”) as well as the corresponding polymer-moiety conjugate.

Nonlimiting examples of GM-CSF moieties include the following: a human GM-CSF as identified in any of SEQ ID NO: 1, SEQ ID NO: 2 and SEQ ID NO: 3; truncated versions thereof; hybrid variants, and peptide mimetics having GM-CSF activity. Biologically active fragments, deletion variants, substitution variants or addition variants of any of the foregoing that maintain at least some degree of GM-CSF activity can also serve as a GM-CSF moiety.

Depending on the system used to express proteins having GM-CSF activity, the GM-CSF moiety can be unglycosylated or glycosylated and either may be used. That is, the GM-CSF moiety can be unglycosylated or the GM-CSF moiety can be glycosylated.

The Water-Soluble Polymer

As previously discussed, each conjugate comprises a GM-CSF attached, either directly or through a spacer moiety comprised of one or more atoms, to a water-soluble polymer. With respect to the water-soluble polymer, the water-soluble polymer is nonpeptidic, nontoxic, non-naturally occurring and biocompatible. With respect to biocompatibility, a substance is considered biocompatible if the beneficial effects associated with use of the substance alone or with another substance (e.g., an active agent such a GM-CSF moiety) in connection with living tissues (e.g., administration to a patient) outweighs any deleterious effects as evaluated by a clinician, e.g., a physician. With respect to non-immunogenicity, a substance is considered nonimmunogenic if the intended use of the substance in vivo does not produce an undesired immune response (e.g., the formation of antibodies) or, if an immune response is produced, that such a response is not deemed clinically significant or important as evaluated by a clinician. It is particularly preferred that the water-soluble polymer is biocompatible and nonimmunogenic.

Further the water-soluble polymer is typically characterized as having from 2 to about 300 termini. Examples of such polymers include, but are not limited to, poly(alkylene glycols) such as polyethylene glycol (PEG), poly(propylene glycol) (“PPG”), copolymers of ethylene glycol and propylene glycol and the like, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and combinations of any of the foregoing.

The polymer is not limited to a particular structure and can be linear (e.g., alkoxy PEG or bifunctional PEG), or non-linear such as branched, forked, multi-armed (e.g., PEGs attached to a polyol core), and dendritic. Moreover, the internal structure of the polymer can be organized in any number of different patterns and can be selected from the group consisting of homopolymer, alternating copolymer, random copolymer, block copolymer, alternating tripolymer, random tripolymer, and block tripolymer.

The activated PEG and other activated water-soluble polymers (collectively “polymeric reagents”) used to form conjugates with the GM-CSF include an activated functional group appropriate for coupling to a desired site on the GM-CSF moiety. Thus, a polymeric reagent includes a functional group for reaction with the GM-CSF moiety. Representative polymeric reagents and methods for conjugating these polymers to an active moiety are known in the art and further described in Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182.

Typically, the weight-average molecular weight of the water-soluble polymer in the conjugate is from about 100 Daltons to about 150,000 Daltons. Exemplary ranges, however, include weight-average molecular weights in the range of greater than 5,000 Daltons to about 150,000 Daltons, in the range of greater than 5,000 Daltons to about 100,000 Daltons, in the range of from about 6,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 15,000 Daltons to about 85,000 Daltons, in the range of from about 20,000 Daltons to about 85,000 Daltons, in the range of from about 20,000 Daltons to about 60,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. For any given water-soluble polymer, PEGs having a molecular weight in one or more of these ranges are preferred.

Exemplary weight-average molecular weights for the water-soluble polymer include about 100 Daltons, about 200.Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. Branched versions of the water-soluble polymer (e.g., a branched 40,000 Dalton water-soluble polymer comprised of two 20,000 Dalton polymers) having a total molecular weight of any of the foregoing can also be used. In one or more embodiments, the conjugate will not have any PEG moieties attached, either directly or indirectly, with a PEG having a weight-average molecular weight of less than about 6,000 Daltons.

When used as the polymer, PEGs will typically comprise a number of (OCH₂CH₂) monomers [or (CH₂CH₂O) monomers, depending on how the PEG is defined]. As used throughout the description, the number of repeating units is identified by the subscript “n” in, for example, “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

With regard to the molecular weight of the water-soluble polymer, in or more embodiments of the invention, a conjugate is provided, the conjugate comprising a GM-CSF moiety covalently attached, either directly or through a spacer moiety comprised of one or more atoms, to a water-soluble polymer, wherein the molecular weight of the water-soluble polymer is greater than 5,000 Daltons.

One particularly preferred polymer for use in the invention is an end-capped polymer, that is, a polymer having at least one terminus capped with a relatively inert group, such as a lower alkoxy group (i.e., a C₁₋₆ alkoxy group), although a hydroxyl group can also be used. When the polymer is PEG, for example, it is preferred to use a methoxy-PEG (commonly referred to as mPEG), which is a linear form of PEG wherein one terminus of the polymer has a methoxy (—OCH₃) group, while the other terminus is a hydroxyl or other functional group that can be optionally chemically modified.

In one form useful in the present invention, free or unbound PEG is a linear polymer terminated at each end with hydroxyl groups:
HO—CH₂CH₂O—(CH₂CH₂O )ₙ—CH₂CH₂—OH,
wherein (n) typically ranges from zero to about 4,000.

The above polymer, alpha-, omega-dihydroxylpoly(ethylene glycol), can be represented in brief form as HO—PEG—OH where it is understood that the —PEG— symbol can represent the following structural unit:
—CH₂CH₂O—(CH₂CH₂O)ₙ—CH₂CH₂—,
wherein (n) is as defined as above.

Another type of PEG useful in the present invention is methoxy-PEG—OH, or mPEG in brief, in which one terminus is the relatively inert methoxy group, while the other terminus is a hydroxyl group. The structure of mPEG is given below.
CH₃O—CH₂CH₂O —(CH₂CH₂O)ₙ—CH₂CH₂—OH
wherein (n) is as described above.

Multi-armed or branched PEG molecules, such as those described in U.S. Pat. No. 5,932,462, can also be used as the PEG polymer. For example, PEG can have the structure: 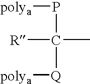
wherein:
polyₐ and poly_{b} are PEG backbones (either the same or different), such as methoxy poly(ethylene glycol);
R″ is a non-reactive moiety, such as H, methyl or a PEG backbone; and
P and Q are non-reactive linkages. In some instances, the branched PEG molecule includes a lysine residue. In some instances, the lysine residue-containing branched PEG reagent will have the following structure (although a succinimidyl-containing structure is shown, reactive groups other than succinimidyl can be replaced therefor): 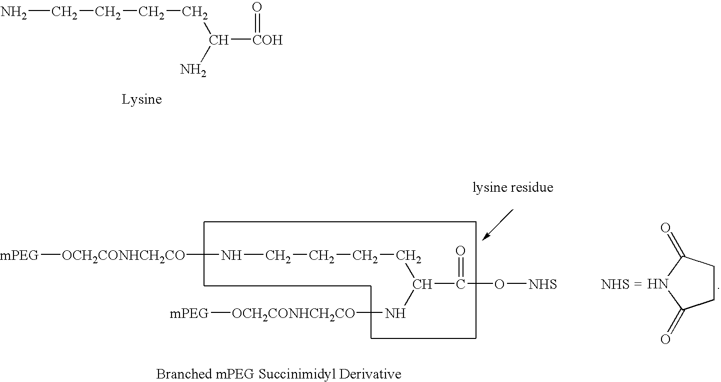
   In some instances, it is preferred that the polymeric reagent (as well as the corresponding conjugate prepared from the polymeric reagent) lacks a lysine residue in which the polymeric portions are connected to amine groups of the lysine via a “—OCH₂CONHCH₂CO—” group. In still other instances, it is preferred that the polymeric reagent (as well as the corresponding conjugate prepared from the polymeric reagent) lacks a branched water-soluble polymer that includes a lysine residue (wherein the lysine residue is used to effect branching).

In addition, the PEG can comprise a forked PEG. An example of a forked PEG is represented by the following structure: 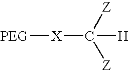
wherein X is a spacer moiety of one or more atoms and each Z is an activated terminal group linked to the carbon atom of C—H by a chain of atoms of defined length. International Application No. PCT/US99/05333 discloses various forked PEG structures capable of use in one or more embodiments of the present invention. The chain of atoms linking the Z functional groups to the branching carbon atom serve as a tethering group and may comprise, for example, alkyl chains, ether chains, ester chains, amide chains and combinations thereof.

The PEG polymer may comprise a pendant PEG molecule having reactive groups, such as carboxyl, covalently attached along the length of the PEG rather than at the end of the PEG chain. The pendant reactive groups can be attached to the PEG directly or through a spacer moiety, such as an alkylene group.

In addition to the above-described forms of PEG, the polymer can also be prepared with one or more weak or degradable linkages (such as a hydrolytically degradable linkage) in the polymer, including any of the above described polymers. For example, PEG can be prepared with ester linkages in the polymer that are subject to hydrolysis. As shown below, this hydrolysis results in cleavage of the polymer into fragments of lower molecular weight:
—PEG—CO₂—PEG—+H₂O→—PEG—CO₂H+HO—PEG—

Other hydrolytically degradable linkages, useful as a degradable linkage within a polymer backbone, include: carbonate linkages; imine linkages resulting, for example, from reaction of an amine and an aldehyde (see, e.g., Ouchi et al. (1997) Polymer Preprints 38(1):582-3); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; orthoester linkages that are, for example, formed by reaction between a formate and an alcohol; certain amide linkages formed by an amine group, e.g., at an end of a polymer such as PEG, and a carboxyl group of another PEG chain; urethane linkages formed from reaction of, e.g., a PEG with a terminal isocyanate group and a PEG alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′ hydroxyl group of an oligonucleotide.

The presence of one or more degradable linkages into-the polymer chain may provide for additional control over the final desired pharmacological properties of the conjugate upon administration. For example, a large and relatively inert conjugate (e.g., having one or more high molecular weight PEG chains attached to a GM-CSF moiety, for example, one or more PEG chains having a molecular weight greater than about 20,000, wherein the conjugate possesses essentially no bioactivity) may be administered, which is hydrolyzed to generate a bioactive conjugate possessing a portion of the original PEG chain. In this way, the properties of the conjugate can be more effectively tailored to balance the bioactivity of the conjugate over time.

Those of ordinary skill in the art will recognize that the foregoing discussion concerning substantially water-soluble polymer segments is by no means exhaustive and is merely illustrative, and that all polymeric materials having the qualities described above are contemplated. As used herein, the term “polymeric reagent” generally refers to an entire molecule, which can comprise a water-soluble polymer segment and a functional group.

Conjugates

As described above, a conjugate of the invention comprises a water-soluble polymer covalently attached (either directly or through a spacer moiety) to a GM-CSF moiety. Typically, for any given conjugate, there will be one to four water-soluble polymers covalently attached to a GM-CSF moiety (wherein for each water-soluble polymer, the water-soluble polymer can be attached either directly to the GM-CSF moiety or through a spacer moiety). In some instances, however, the conjugate may have 1, 2, 3, 4, 5, 6, 7, 8 or more water-soluble polymers individually attached to a GM-CSF moiety (again, with respect to each water-soluble polymer, attached directly or through a spacer moiety). In addition, the conjugate may include not more than 8 water-soluble polymers individually attached to a GM-CSF moiety, not more than 7 water-soluble polymers individually attached to a GM-CSF moiety, not more than 6 water-soluble polymers individually attached to a GM-CSF moiety, not more than 5 water-soluble polymers individually attached to a GM-CSF moiety, not more than 4 water-soluble polymers individually attached to a GM-CSF moiety, not more than 3 water-soluble polymers individually attached to a GM-CSF moiety, not more than 2 water-soluble polymers individually attached to a GM-CSF moiety, and not more than 1 water-soluble polymer attached to a GM-CSF moiety.

The particular linkage between the GM-CSF moiety and the water-soluble polymer (or the spacer moiety that is attached to the water-soluble polymer) depends on a number of factors. Such factors include, for example, the particular linkage chemistry employed, the particular GM-CSF moiety, the available functional groups within the GM-CSF moiety (either for attachment to a polymer or conversion to a suitable attachment site), the possible presence of additional reactive functional groups within the GM-CSF moiety, and the like.

In one or more embodiments of the invention, the linkage between the GM-CSF moiety and the polymer (or the spacer moiety that is attached to the polymer) is a hydrolytically stable linkage, such as an amide, urethane (also known as carbamate), amine, thioether (also known as sulfide), or urea (also known as carbamide). In one or more embodiments, the linkage does not result from reaction of the polymeric reagent bearing a functional group with the GM-CSF moiety, wherein the functional group is selected from the group consisting of triazine, hydrazine, hydrazide, aldehyde, semicarbazide, maleimide, vinylsulfone, phenylglyoxal, isocyanate, isothiocyanate, amine and tresyl functional group with the GM-CSF moiety.

In one or more embodiments of the invention, the linkage between the GM-CSF moiety and the water-soluble polymer (or the spacer moiety that is attached to the water-soluble polymer) is a degradable linkage. In this way, the linkage linking the GM-CSF moiety is “degradable.” That is, the water-soluble polymer (and the spacer moiety, when present) cleaves (either through hydrolysis, enzymatic processes, or otherwise), thereby resulting in the native or an unconjugated GM-CSF moiety. Preferably, degradable linkages result in the water-soluble polymer (and any spacer moiety) detaching from the GM-CSF moiety in vivo without leaving any fragment of the water-soluble polymer (and any spacer moiety). Exemplary degradable linkages include carbonate, carboxylate ester, phosphate ester, thiolester, anhydrides, acetals, ketals, acyloxyalkyl ether, imines, and orthoesters. Such linkages can be readily prepared by appropriate modification of either the GM-CSF moiety (e.g., the carboxyl group C terminus of the protein or a side chain hydroxyl group of an amino acid such as serine or threonine contained within the protein) and/or the polymeric reagent using coupling methods commonly employed in the art. Most preferred, however, are hydrolyzable linkages that are readily formed by reaction of a suitably activated polymer with a non-modified functional group contained within the GM-CSF moiety.

With regard to linkages, in one more embodiments of the invention, a conjugate is provided, comprising a GM-CSF moiety covalently attached at an amino acid residue, either directly or through a spacer moiety comprised of one or more atoms, to a water-soluble polymer.

The conjugates (as opposed to an unconjugated GM-CSF moiety) may or may not possess a measurable degree of GM-CSF activity. That is to say, a conjugate in accordance with the invention will possesses anywhere from about 0% to about 100% or more of the bioactivity of the unmodified parent GM-CSF moiety. Preferably, compounds possessing little or no GM-CSF activity typically contain a degradable linkage connecting the polymer to the moiety, so that regardless of the lack of activity in the conjugate, the active parent molecule (or a derivative thereof having GM-CSF activity) is released by degradation of the linkage (e.g., hydrolysis upon aqueous-induced cleavage of the linkage). Such activity may be determined using a suitable in vivo or in vitro model, depending upon the known activity of the particular moiety having GM-CSF activity employed.

Optimally, degradation of a degradable linkage is facilitated through the use of hydrolytically cleavable and/or enzymatically degradable linkages such as urethane, amide, carbonate or ester-containing linkages. In this way, clearance of the conjugate [via cleavage of individual water-soluble polymer(s)] can be modulated by selecting the polymer molecular size and the type of functional group that would provide the desired clearance properties. One of ordinary skill in the art can determine the proper molecular size of the polymer as well as the cleavable functional group. For example, one of ordinary skill in the art, using routine experimentation, can determine a proper molecular size and cleavable functional group by first preparing a variety of polymer-(GM-CSF) conjugates with different weight-average molecular weights and degradable functional groups, and then obtaining the clearance profile for each conjugate by administering the conjugate to a patient and taking periodic blood and/or urine sampling. Once a series of clearance profiles has been obtained for each tested conjugate, a conjugate having the desired clearance profile can be determined.

For conjugates possessing a hydrolytically stable linkage that couples the GM-CSF moiety to the water-soluble polymer, the conjugate will typically possess a measurable degree of GM-CSF activity. For instance, such conjugates are typically characterized as having a bioactivity satisfying one or more of the following percentages relative to that of the unconjugated GM-CSF moiety: at least about 2%, at least about 5%, at least about 10%, at least about 15%, at least about 25%, at least about 30%, at least about 40%, at least about 50%, at least about 60%, at least about 80%, at least about 85%, at least about 90%, at least about 95%, at least about 97%, at least about 100%, and more than 105% (when measured in a suitable model, such as those presented here and/or well known in the art). Preferably, conjugates having a hydrolytically stable linkage (e.g., an amide linkage) will possess at least some degree of the bioactivity of the unmodified parent GM-CSF moiety.

Exemplary conjugates will now be described. The GM-CSF moiety is expected to share (at least in part) an amino acid sequence similar or related to a human GM-CSF. Thus, as previously indicated, while reference will be made to specific locations or atoms within a human GM-CSF, such a reference is for convenience only and one having ordinary skill in the art will be able to readily determine the corresponding location or atom in other moieties having GM-CSF activity. In particular, the description provided herein for a human GM-CSF is often applicable not only to a human GM-CSF, but to fragments, deletion variants, substation variants and addition variants of any of the foregoing.

Amino groups on GM-CSF moieties provide a point of attachment between the GM-CSF moiety and the water-soluble polymer. Each of the human GM-CSF moieties provided in SEQ ID NO: 1, SEQ ID NO: 2 and SEQ ID NO: 3, comprises 14 lysine residues, each lysine residue containing an ε-amino group that may be available for conjugation, as well as one amino terminus. See SEQ ID NO: 1 and SEQ ID NO: 2 and SEQ ID NO: 3. Thus, exemplary attachment points include attachment at an amino acid (through the amine-containing side chain of a lysine residue) at any one or more of positions 25, 26, 28, 49, 55, 59, 61, 66, 64, 73, 77, 110, 114 and 115. Additionally, another GM-CSF moiety contains 15 amine-containing lysine residues (see SEQ ID NO: 3). Thus, preferred attachment points of this GM-CSF include attachment at the amine residue associated with a lysine at any one of positions 23, 25, 26, 28, 49, 55, 59, 61, 66, 64, 73, 77, 110, 114 and 115.

There are a number of examples of suitable water-soluble polymeric reagents useful for forming covalent linkages with available amines of a GM-CSF moiety. Specific examples, along with the corresponding conjugates, are provided in Table 1, below. In the table, the variable (n) represents the number of repeating monomeric units and “(GM-CSF)” represents the GM-CSF moiety following conjugation to the water-soluble polymer. While each polymeric portion [e.g., (OCH₂CH₂)ₙ or (CH₂CH₂O)ₙ] presented in Table 1 terminates in a “CH₃” group, other groups (such as H and benzyl) can be substituted therefor.

TABLE 1
Amine-Specific Polymeric Reagents and the GM-CSF Moiety Conjugate Formed Therefrom
Polymeric Reagent

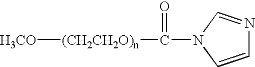

mPEG-Oxycarbonylimidazole Reagent

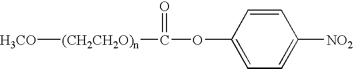

mPEG Nitrophenyl Reagent

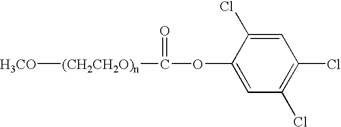

mPEG-Trichlorophenyl Carbonate Reagent

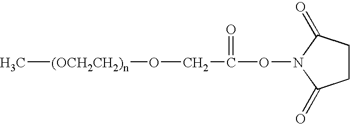

mPEG-Succinimidyl Reagent

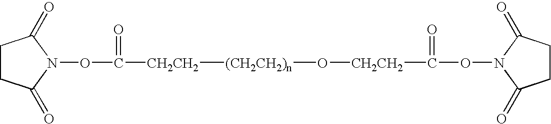

Homobifunctional PEG-Succinimidyl Reagent

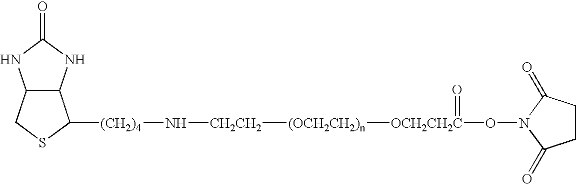

Heterobifunctional PEG-Succinimidyl Reagent

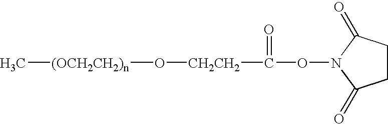

mPEG-Succinimidyl Reagent

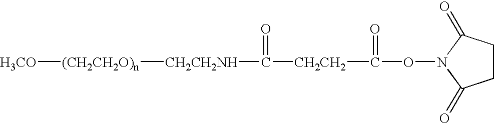

mPEG-Succinimdyl Reagent

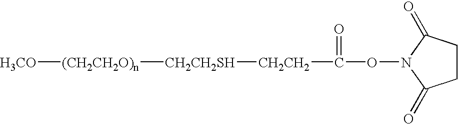

mPEG-Succinimidyl Reagent

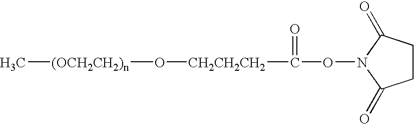

mPEG-Succinimidyl Reagent

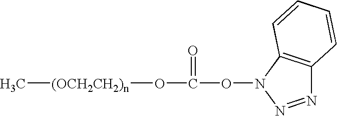

mPEG-Benzotriazole Carbonate Reagent

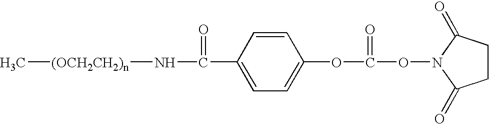

mPEG-Succinimidyl Reagent

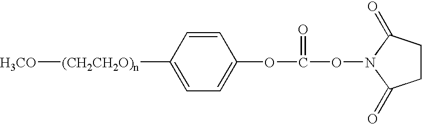

mPEG-Succinimidyl Reagent

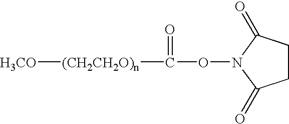

mPEG-Succinimidyl Reagent

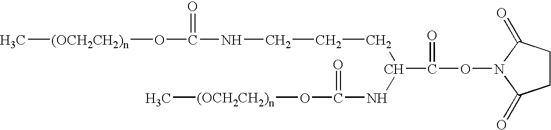

Branched mPEG2-N-Hydroxysuccinimide Reagent

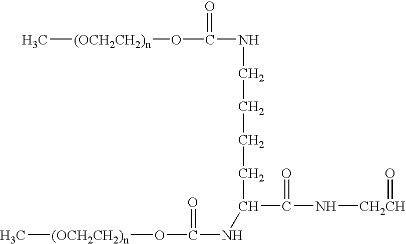

Branched mPEG2-Aldehyde Reagent

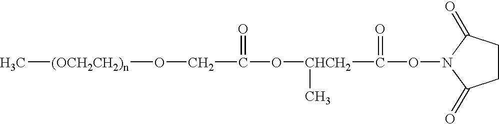

mPEG-Succinimidyl Reagent

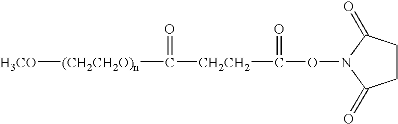

mPEG-Succinimidyl Reagent

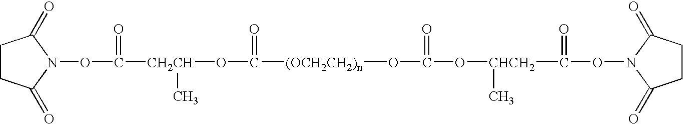

Homobifunctional PEG-Succinimidyl Reagent

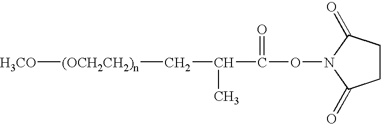

mPEG-Succinimidyl Reagent

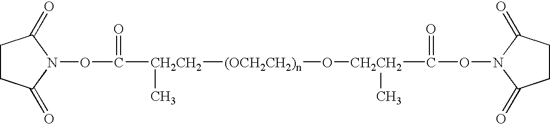

Homobifunctional PEG-Succinimidyl Propionate
Reagent

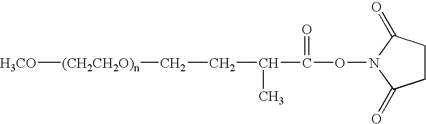

mPEG-Succinimidyl Reagent

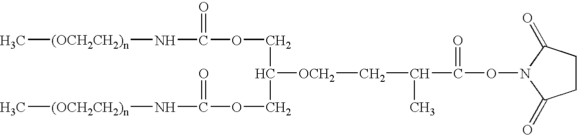

Branched mPEG2-N-Hydroxysuccinimide Reagent

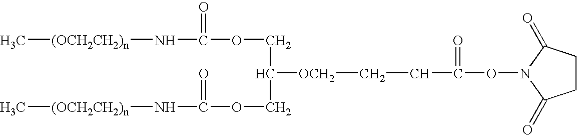

Branched mPEG2-N-Hydroxysuccinimide Reagent

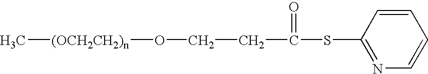

mPEG-Thioester Reagent

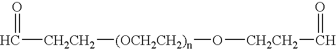

Homobifunctional PEG Propionaldehyde Reagent

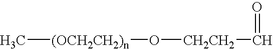

mPEG Propionaldehyde Reagent

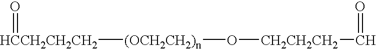

Homobifunctional PEG Butyraldehyde Reagent

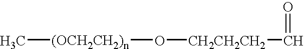

mPEG Butryaldehyde Reagent

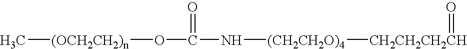

mPEG Butryaldehyde Reagent

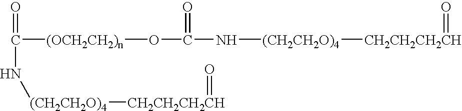

Homobifunctional PEG Butyraldehyde Reagent

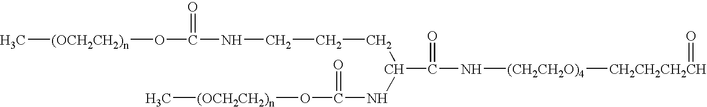

Branched mPEG2-N-Hydroxysuccinimide Reagent

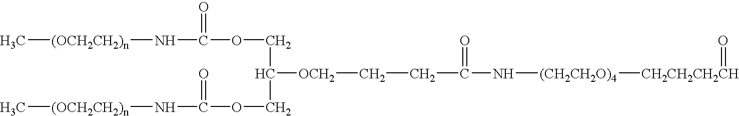

Branched mPEG2-N-Hydroxysuccinimide Reagent

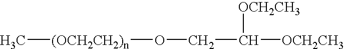

mPEG Acetal Reagent

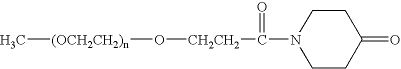

mPEG Piperidone Reagent

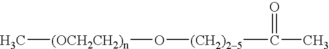

mPEG Methylketone Reagent

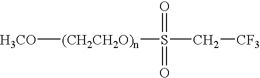

mPEG Tresylate Reagent

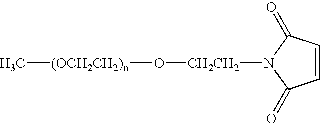

mPEG Maleimide Reagent
(under certain reaction conditions such as pH > 8)

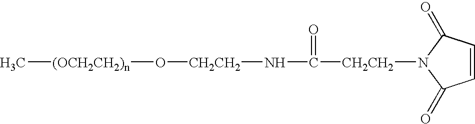

mPEG Maleimide Reagent
(under certain reaction conditions such as pH > 8)

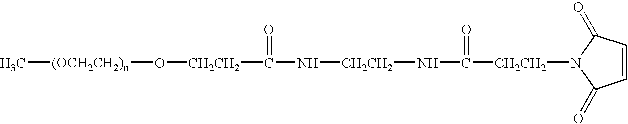

mPEG Maleimide Reagent
(under certain reaction conditions such as pH > 8)

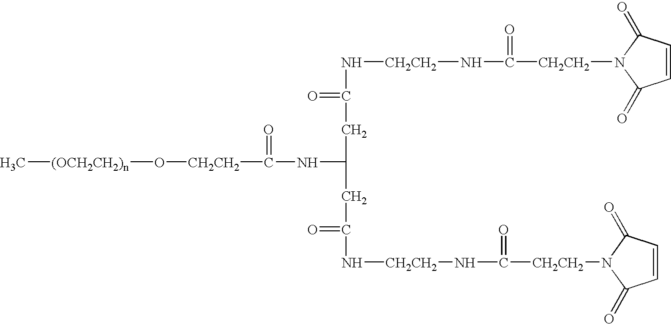

mPEG Forked Maleimide Reagent
(under certain reaction conditions such as pH > 8)

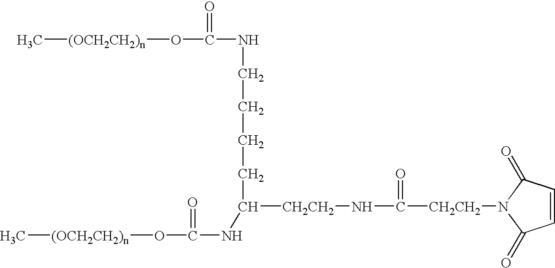

branched mPEG Maleimide Reagent
(under certain reaction conditions such as pH > 8)
Corresponding Conjugate

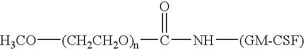

Carbamate Linkage

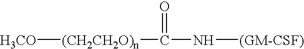

Carbamate Linkage

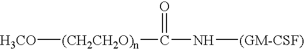

Carbamate Linkage

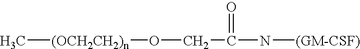

Amide Linkage

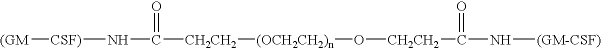

Amide Linkages

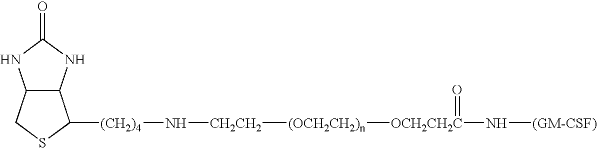

Amide Linkage

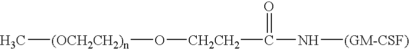

Amide Linkage

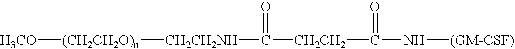

Amide Linkage

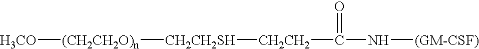

Amide Linkage

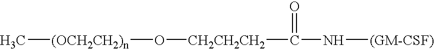

Amide Linkage

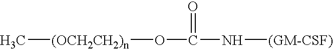

Carbamate Linkage

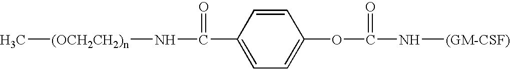

Carbamate Linkage

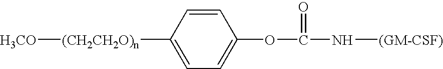

Amide Linkage

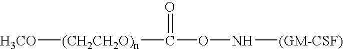

Amide Linkage

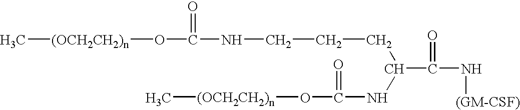

Amide Linkage

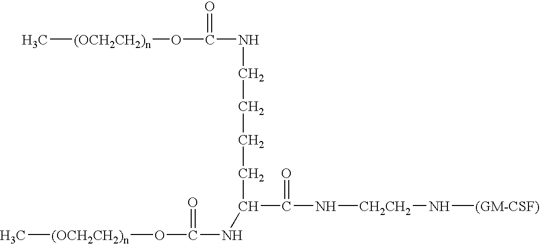

Secondary Amine Linkage

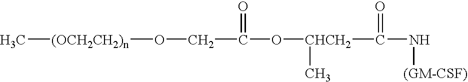

Amide Linkage

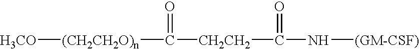

Amide Linkage

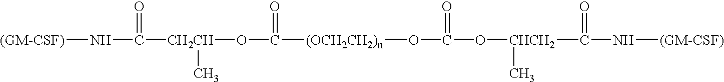

Amide Linkage

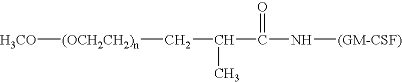

Amide Linkage

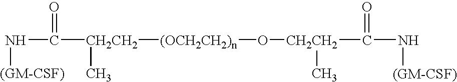

Amide Linkage

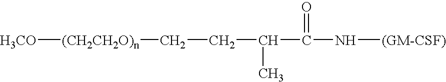

Amide Linkage

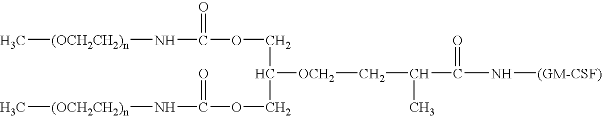

Amide Linkage

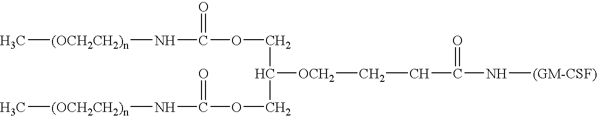

Amide Linkage

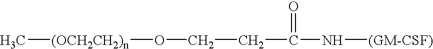

Amide Linkage (typically to a GM-CSF moiety
having an N-terminal cysteine or histidine)

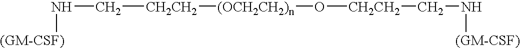

Secondary Amine Linkages

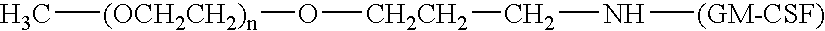

Secondary Amine Linkages

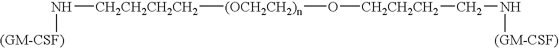

Secondary Amine Linkages

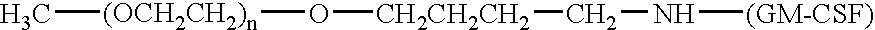

Secondary Amine Linkages

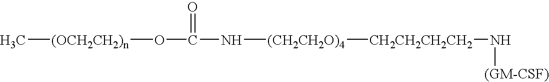

Secondary Amine Linkages

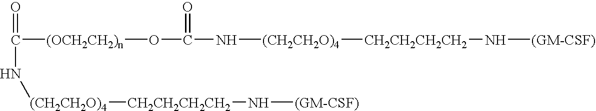

Secondary Amine Linkages

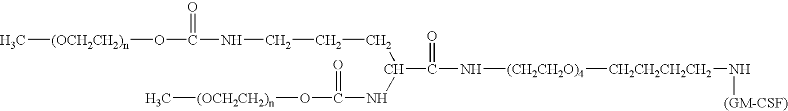

Secondary Amine Linkages

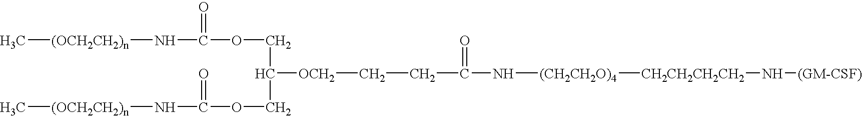

Secondary Amine Linkages

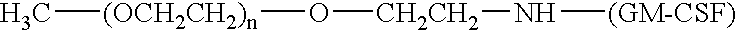

Secondary Amine Linkages

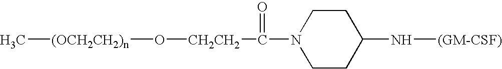

Secondary Amine Linkages
(to a secondary carbon)

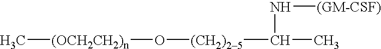

secondary amine linkage
(to a secondary carbon)

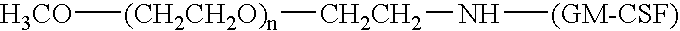

Secondary Amine Linkage

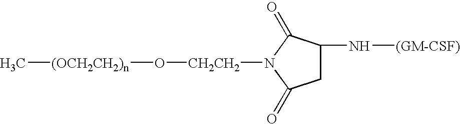

Secondary Amine Linkage

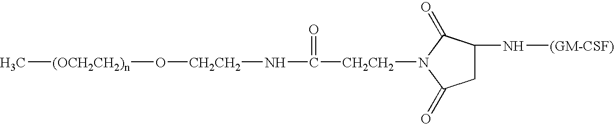

Secondary Amine Linkage

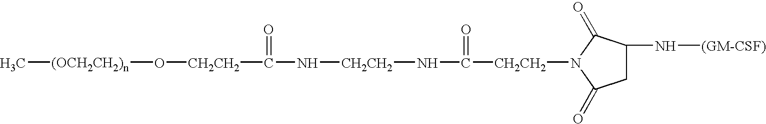

Secondary Amine Linkage

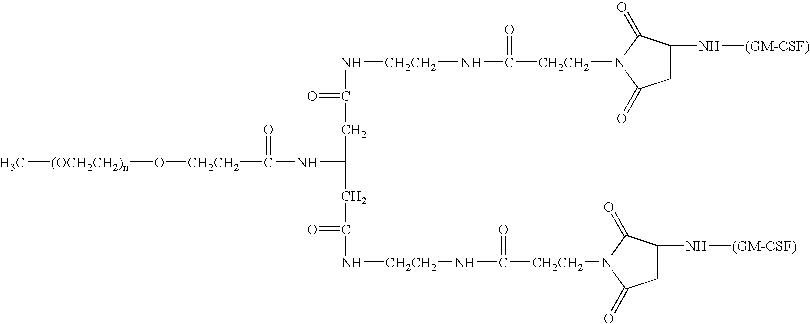

Secondary Amine Linkage

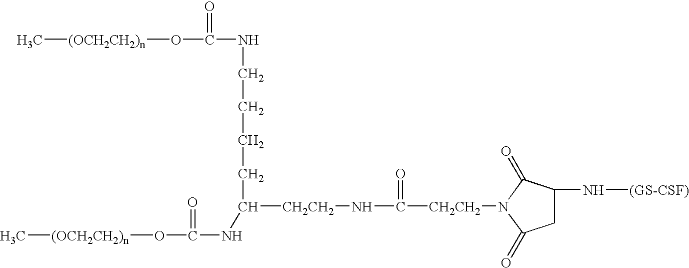

Secondary Amine Linkage

Conjugation of a polymeric reagent to an amine group of a GM-CSF moiety can be accomplished by a variety of techniques. In one approach, a GM-CSF moiety can be conjugated to a polymeric reagent functionalized with a succinimidyl derivative (or other activated ester group, wherein approaches similar to those described for a succinimidyl derivative can be used for other activated ester group-containing polymeric reagents). In this approach, the polymeric reagent bearing a succinimidyl group can be attached to the GM-CSF moiety in aqueous media at a pH of 7.0 to 9.0, although different reaction conditions (e.g., a lower pH such as 6 to 7, or different temperatures and/or less than 15° C.) can result in the attachment of a polymer to a different location on the GM-CSF moiety.

Exemplary conjugates that can be prepared using, for example, polymeric reagents containing a reactive ester comprise the following structure 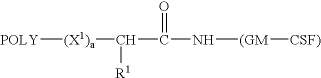
wherein:
POLY is a water-soluble polymer,
(a) is either zero or one;
X¹, when present, is a spacer moiety comprised of one or more atoms;
R¹ is hydrogen an organic radical; and
GM-CSF is a GM-CSF moiety.

With respect to the structure corresponding to that referred to in the immediately preceding paragraph, any of the water-soluble polymers provided herein can be defined as POLY, any of the spacer moieties provided herein can be defined as X¹ (when present), any of the organic radicals provided herein can be defined as R¹ (in instances where R¹ is not hydrogen), and any of the GM-CSF moieties provided herein can be defined as GM-CSF. With respect to the structure corresponding to that referred to in the immediately preceding paragraph, it is preferred that: POLY is a poly(ethylene glycol) such as H₃CO(CH₂CH₂O )ₙ—, wherein (n) is an integer having a value of from 3 to 4000; (a) is one; X¹ is a C₁₋₆ alkylene, more preferably selected from the group consisting of methylene (i.e., —CH₂—), ethylene (i.e., —CH₂—CH₂—) and propylene (i.e., —CH₂—CH₂—CH₂—); R¹ is H or lower alkyl such as methyl or ethyl; and GM-CSF is a human GM-CSF.

Typical of another approach useful for conjugating a GM-CSF moiety to a polymeric reagent is the use of a reductive amination reaction to conjugate a primary amine of a GM-CSF moiety with a polymeric reagent functionalized with a ketone, aldehyde or a hydrated form thereof (e.g., ketone hydrate and aldehyde hydrate). In this approach, the primary amine from the GM-CSF moiety reacts with the carbonyl group of the aldehyde or ketone (or the corresponding hydroxy-containing group of a hydrated aldehyde or ketone), thereby forming a Schiff base. The Schiff base, in turn, can then be reductively converted to a stable conjugate through use of a reducing agent such as sodium borohydride. Selective reactions (e.g., at the N-terminus are possible) are possible, particularly with a polymer functionalized with a ketone or an alpha-methyl branched aldehyde and/or under specific reaction conditions (e.g., reduced pH).

Exemplary conjugates that can be prepared using, for example, polymeric reagents containing an aldehyde (or aldehyde hydrate) or ketone or (ketone hydrate) comprise the following structure: 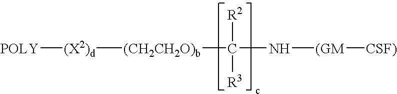
wherein:
POLY is a water-soluble polymer;
(d) is either zero or one;
X², when present, is a spacer moiety comprised of one or more atoms;
(b) is an integer having a value of one through ten;
(c) is an integer having a value of one through ten;
R², in each occurrence, is independently H or an organic radical;
R³, in each occurrence, is independently H or an organic radical; and
GM-CSF is a GM-CSF moiety.

With respect to the structure corresponding to that referred to in immediately preceding paragraph, any of the water-soluble polymers provided herein can be defined as POLY, any of the spacer moieties provided herein can be defined as X² (when present), any of the organic radicals provided herein can be independently defined as R² and R³ (in instances where R² and R³ are independently not hydrogen), and any of the GM-CSF moieties provided herein can be defined as GM-CSF. With respect to the structure corresponding to that referred to in the immediately preceding paragraph. it is preferred in some instances that: POLY is a poly(ethylene glycol) such as H₃CO(CH₂CH₂O)ₙ—, wherein (n) is an integer having a value of from 3 to 4000; (d) is one; X¹ is amide [e.g., —C(O)NH—]; (b) is 2 through 6, more preferably 4; (c) is 2 through 6, more preferably 4; each of R² and R³ are independently H or lower alkyl, more preferably methyl when lower alkyl; and GM-CSF is a human GM-CSF. In other instances, it is preferred that the conjugate comprises the following structure: 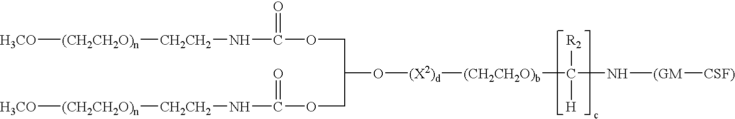
wherein:
each (n) is independently an integer having a value of from 3 to 4000;
X² is as previously defined;
(b) is 2 through 6;
(c) is 2 through 6;
R², in each occurrence, is independently H or lower alkyl; and
GM-CSF is a GM-CSF moiety.

Carboxyl groups represent another functional group that can serve as a point of attachment on the GM-CSF moiety. Structurally, the conjugate will comprise the following: 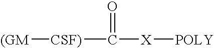
where GM-CSF and the adjacent carbonyl group correspond to the carboxyl-containing GM-CSF moiety, X is a spacer moiety, preferably a heteroatom selected from O, N(H), and S, and POLY is a water-soluble polymer such as PEG, optionally terminating in an end-capping moiety.

The C(O)—X linkage results from the reaction between a polymeric derivative bearing a terminal functional group and a carboxyl-containing GM-CSF moiety. As discussed above, the specific linkage will depend on the type of functional group utilized. If the polymer is end-functionalized or “activated” with a hydroxyl group, the resulting linkage will be a carboxylic acid ester and X will be O. If the polymer backbone is functionalized with a thiol group, the resulting linkage will be a thioester and X will be S. When certain multi-arm, branched or forked polymers are employed, the C(O)X moiety, and in particular the X moiety, may be relatively more complex and may include a longer linkage structure.

Polymeric reagents containing a hydrazide moiety are also useful for conjugation at a carbonyl. To the extent that the GM-CSF moiety does not contain a carbonyl moiety, a carbonyl moiety can be introduced by reducing any carboxylic acids (e.g., the C-terminal carboxylic acid) and/or by providing a glycosylated version (wherein the added sugar has a carbonyl moiety) of the GM-CSF moiety. Specific examples of polymeric reagents comprising a hydrazide moiety, along with the corresponding conjugates, are provided in Table 2, below. In addition, any polymeric reagent comprising an activated ester (e.g., a succinimidyl group) can be converted to contain a hydrazide moiety by reacting the polymeric reagent comprising the activated ester with hydrazine (NH₂—NH₂) or tert-butyl carbazate [NH₂NHCO₂C(CH₃)₃]. In the table, the variable (n) represents the number of repeating monomeric units and “═C—(GM-CSF)” represents a GM-CSF moiety following conjugation to the polymeric reagent. Optionally, the hydrazone linkage can be reduced using a suitable reducing agent. While each polymeric portion [e.g., (OCH₂CH₂)ₙ or (CH₂CH₂O )ₙ] presented in Table 2 terminates in a “CH₃” group, other groups (such as H and benzyl) can be substituted therefor.

TABLE 2
Carboxyl-Specific Polymeric Reagents and the GM-CSF Moiety Conjugate Formed Therefrom
Polymeric Reagent

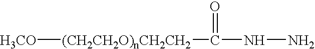

mPEG-Hydrazine Reagent

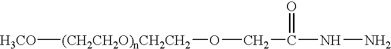

mPEG-Hydrazine Reagent

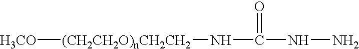

mPEG-Hydrazine Reagent

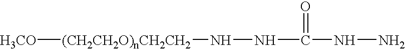

mPEG-Hydrazine Reagent

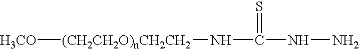

mPEG-Hydrazine Reagent

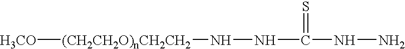

mPEG-Hydrazine Reagent

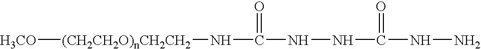

mPEG-Hydrazine Reagent

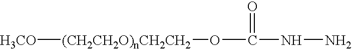

mPEG-Hydrazine Reagent
Corresponding Conjugate

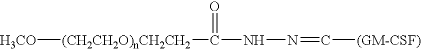

Hydrazone Linkage

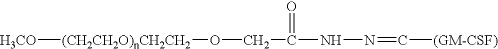

Hydrazone Linkage

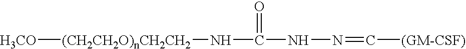

Hydrazone Linkage

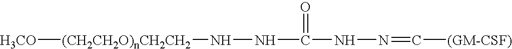

Hydrazone Linkage

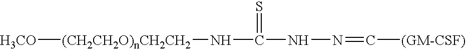

Hydrazone Linkage

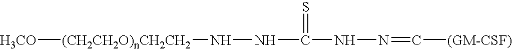

Hydrazone Linkage

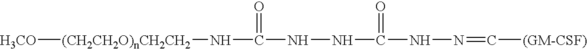

Hydrazone Linkage

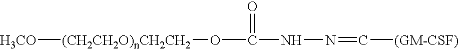

Hydrazone Linkage

Thiol groups contained within the GM-CSF moiety can serve as effective sites of attachment for the water-soluble polymer. The thiol groups in cysteine residues of the GM-CSF moiety can be reacted with an activated PEG that is specific for reaction with thiol groups, e.g., an N-maleimidyl polymer or other derivative, as described in, for example, U.S. Pat. No. 5,739,208, in International Patent Publication No. WO 01/62827, and in Table 3 below.

With respect to both SEQ ID NO: 1, SEQ ID NO: 2, SEQ ID NO: 3, there are four thiol-containing cysteine residues. While not wishing to be bound by theory, it is believed that all cysteine residues in these sequences are involved with disulfide bonding. As a consequence, conjugation to a cysteine residue participating in disulfide bonding may disrupt the tertiary structure of a GM-CSF moiety and potentially significantly decrease its overall activity. Thus, to the extent that any particular GM-CSF moiety lacks a thiol group or disruption of disulfide bonds is to be avoided, it is possible to add a cysteine residue to the GM-CSF moiety using conventional synthetic techniques. See, for example, U.S. Pat. No. 6,608,183 and the procedure described in International Patent Publication WO 90/12874, wherein such a procedure can be adapted for a GM-CSF moiety. In addition, conventional genetic engineering processes can also be used to introduce a cysteine residue into the GM-CSF moiety.

Specific examples, along with the corresponding conjugates, are provided in Table 3, below. In the table, the variable (n) represents the number of repeating monomeric units and “—S—(GM-CSF)” represents the GM-CSF moiety following conjugation to the water-soluble polymer. While each polymeric portion [e.g., (OCH₂CH₂)ₙ or (CH₂CH₂O)ₙ] presented in Table 3 terminates in a “CH₃” group, other groups (such as H and benzyl) can be substituted therefor.

TABLE 3
Thiol-Specific Polymeric Reagents and the GM-CSF Moiety Conjugate Formed Therefrom
Polymeric Reagent

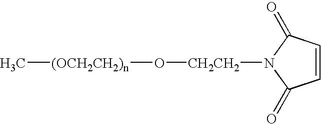

mPEG Maleimide Reagent

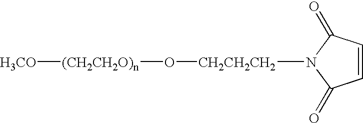

mPEG Maleimide Reagent

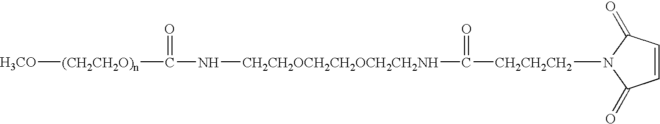

mPEG Maleimide Reagent

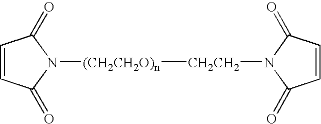

Homobifunctional mPEG Maleimide Reagent

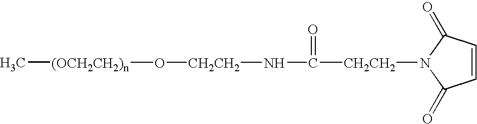

mPEG Maleimide Reagent

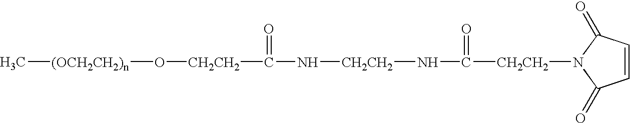

mPEG Maleimide Reagent

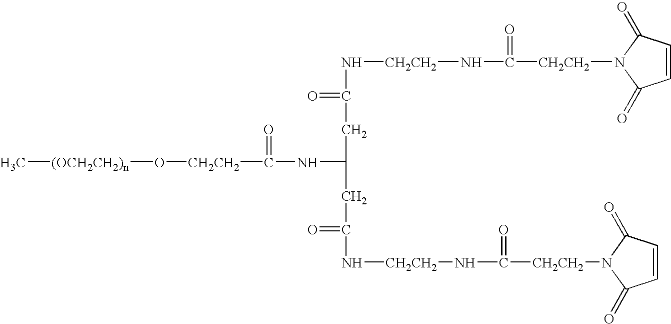

mPEG Forked Maleimide Reagent

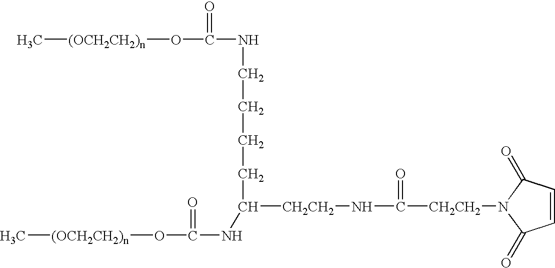

branched mPEG2 Maleimide Reagent

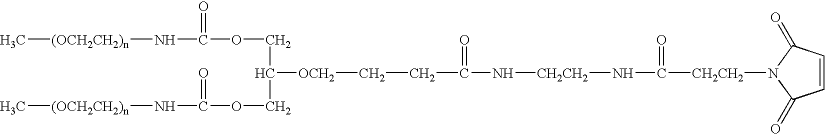

branched mPEG2 Maleimide Reagent

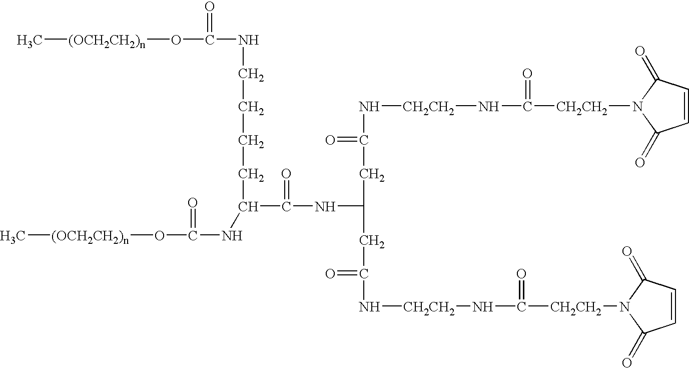

Branched mPEG2 Forked Maleimide Reagent

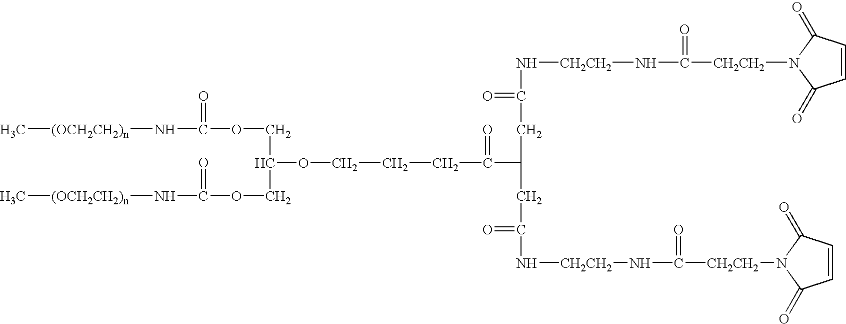

Branched mPEG2 Forked Maleimide Reagent

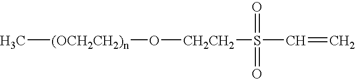

mPEG Vinyl Sulfone Reagent

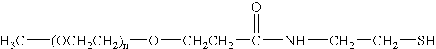

mPEG Thiol Reagent

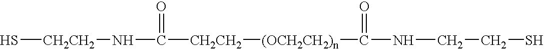

Homobifunctional PEG Thiol Reagent

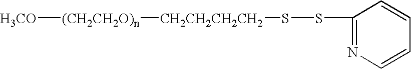

mPEG Disulfide Reagent

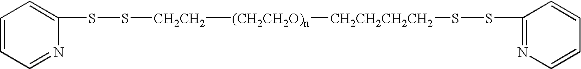

Homobifunctional PEG Disulfide Reagent
Corresponding Conjugate

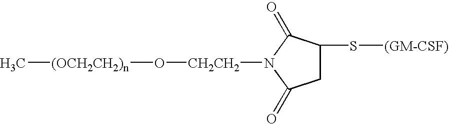

Thioether Linkage

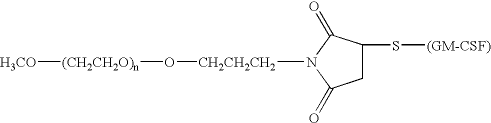

Thioether Linkage

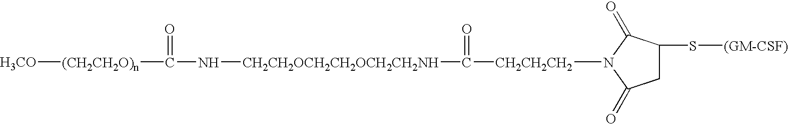

Thioether Linkage

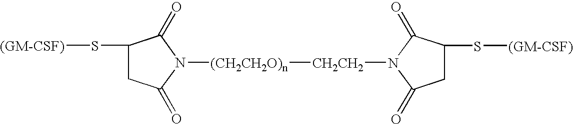

Thioether Linkage

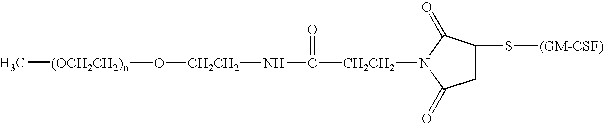

Thioether Linkage

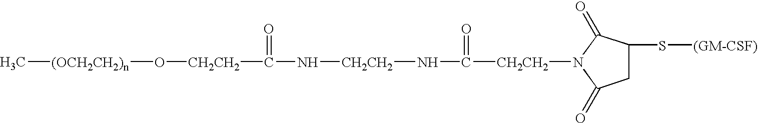

Thioether Linkage

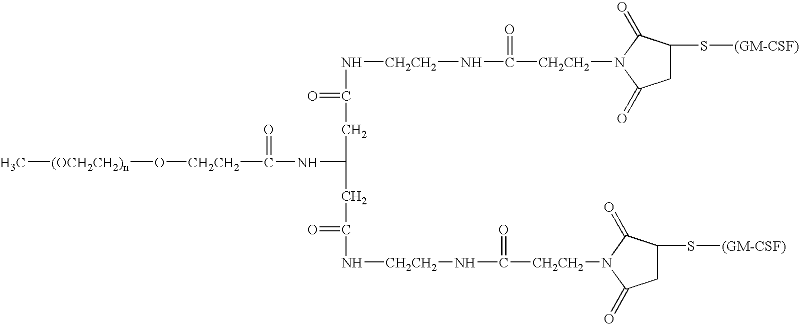

Thioether Linkage

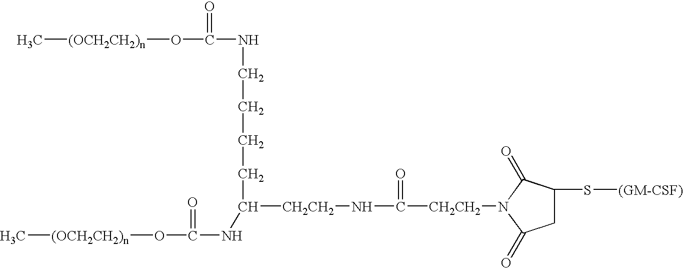

Thioether Linkage

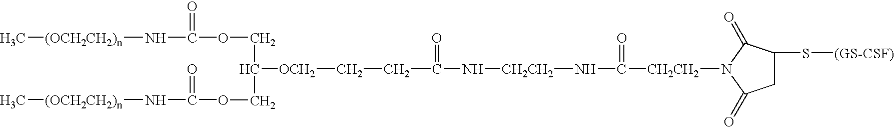

Thioether Linkage

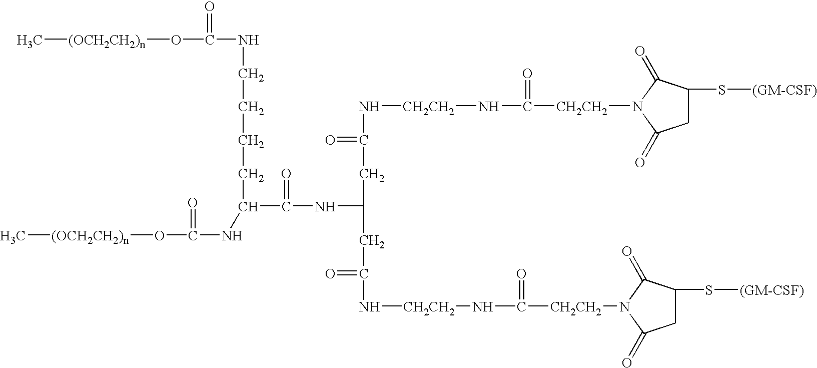

Thioether Linkage

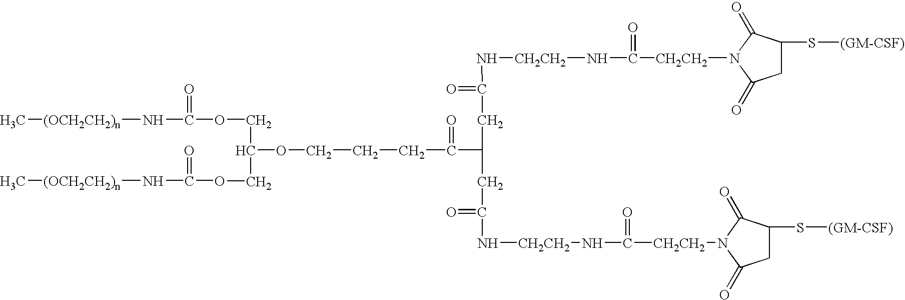

Thioether Linkage

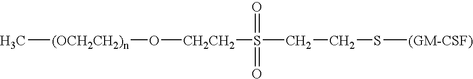

Thioether Linkage

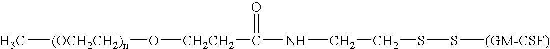

Disulfide Linkage

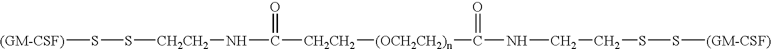

Disulfide Linkage

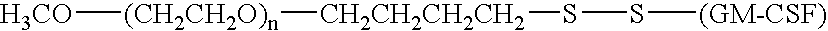

Disulfide Linkage

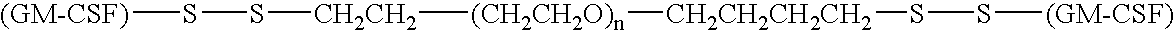

Disulfide Linkage

With respect to conjugates formed from water-soluble polymers bearing one or more maleimide functional groups (regardless of whether the maleimide reacts with an amine or thiol group on the GM-CSF moiety), the corresponding maleamic acid form(s) of the water-soluble polymer can also react with the GM-CSF moiety. Under certain conditions (e.g., a pH of about 7-9 and in the presence of water), the maleimide ring will “open” to form the corresponding maleamic acid. The maleamic acid, in turn, can react with an amine or thiol group of a GM-CSF moiety. Exemplary maleamic acid-based reactions are schematically shown below. POLY represents the water-soluble polymer, and GM-CSF represents the GM-CSF moiety.

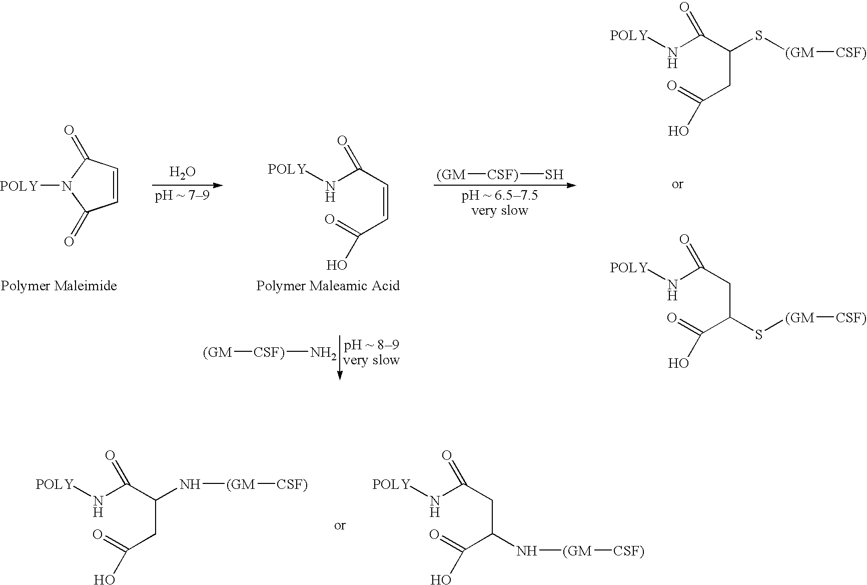

A representative conjugate in accordance with the invention can have the following structure:
POLY-L_{0,1}—C(O)Z—Y—S—S—(GM-CSF)
wherein POLY is a water-soluble polymer, L is an optional linker, Z is a heteroatom selected from the group consisting of O, NH, and S, and Y is selected from the group consisting of C₂₋₁₀ alkyl, C₂₋₁₀ substituted alkyl, aryl, and substituted aryl, and GM-CSF is a GM-CSF moiety. Polymeric reagents that can be reacted with a GM-CSF moiety and result in this type of conjugate are described in U.S. Patent Application Publication No. 2005/0014903.

With respect to polymeric reagents, those described here and elsewhere can be purchased from commercial sources (e.g., Nektar Therapeutics, Huntsville Ala.). In addition, methods for preparing polymeric reagents are described in the literature.

The attachment between the GM-CSF moiety and water-soluble polymer can be direct, wherein no intervening atoms are located between the GM-CSF moiety and the polymer, or indirect, wherein one or more atoms are located between the GM-CSF moiety and polymer. With respect to the indirect attachment, a “spacer moiety” serves as a link between the GM-CSF moiety and the water-soluble polymer. The one or more atoms making up the spacer moiety can include one or more of carbon atoms, nitrogen atoms, sulfur atoms, oxygen atoms, and combinations thereof. The spacer moiety can comprise an amide, secondary amine, carbamate, thioether, and/or disulfide group. Nonlimiting examples of specific spacer moieties (including “X”, X¹ and X²) include those selected from the group consisting of —O—, —S—, —S—S—, —C(O)—, —C(O)—NH—, —NH—C(O)—NH—, —O —C(O)—NH—, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[CH₂]ₕ—(OCH2CH2)ⱼ—, bivalent cycloalkyl group, —O—, —S—, an amino acid, —N(R⁶)—, and combinations of two or more of any of the foregoing, wherein R⁶ is H or an organic radical selected from the group consisting of alkyl, substituted alkyl, alkenyl, substituted alkenyl, alkynyl, substituted alkynyl, aryl and substituted aryl, (h) is zero to six, and (j) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁₋₆ means that the structure can contain 1, 2, 3, 4, 5 or 6 methylenes. Additionally, any of the above spacer moieties may further include an ethylene oxide oligomer chain comprising 1 to 20 ethylene oxide monomer units [i.e., —(CH₂CH₂O)₁₋₂₀]. That is, the ethylene oxide oligomer chain can occur before or after the spacer moiety, and optionally in between any two atoms of a spacer moiety comprised of two or more atoms. Also, the oligomer chain would not be considered part of the spacer moiety if the oligomer is adjacent to a polymer segment and merely represent an extension of the polymer segment. The spacer moiety does not include sugars or carbohydrates (that is, a spacer moiety specifically does not include the sugar of a glycosylate residue). The water-soluble polymer can be attached through a glycosylate residue (e.g., a sugar or carbohydrate). In those instances where such an arrangement is desired, the present application will refer to such an arrangement as “a GM-CSF moiety covalently attached to a water-soluble polymer through a glycosylate residue.”

As indicated above, in some instances the water-soluble polymer-(GM-CSF) conjugate will include a non-linear water-soluble polymer. Such a non-linear water-soluble polymer includes a branched water-soluble polymer (although other non linear water-soluble polymers are also contemplated). Thus, in one or more embodiments of the invention, the conjugate comprises a GM-CSF moiety comprising an internal amine covalently attached, either directly or through a spacer moiety comprised of one or more atoms, to a branched water-soluble polymer. As used herein, an internal amine is an amine that is not part of the N-terminal amino acid (and thus includes not only the N-terminal amine, but any amine on the side chain of the N-terminal amino acid). With respect to a GM-CSF moiety having a sequence of comprising SEQ ID NO: 1, SEQ ID NO: 2, and SEQ ID NO: 3, for example, internal amines are located on each of the chain of each lysine residues.

It must be noted that although such conjugates include a branched water-soluble polymer attached (either directly or through a spacer moiety) to a GM-CSF moiety at an internal amino acid of the GM-CSF moiety, additional branched water-soluble polymers can also be attached to the same GM-CSP moiety at other locations as well. Thus, for example, a conjugate including a branched water-soluble polymer attached (either directly or through a spacer moiety) to a GM-CSF moiety at an internal amino acid of the GM-CSF moiety, can further include an additional branched water-soluble polymer covalently attached, either directly or through a spacer moiety comprised of one or more atoms, to the N-terminal amino acid residue, preferably at the N-terminal amine. As stated above, in some instances, the branched water-soluble polymer lacks a lysine residue in which the polymeric portions are connected to amine groups of the lysine via a “—OCH₂CONHCH₂CO—” group. In still other instances, it is preferred that the branched water-soluble polymer lacks a lysine residue (wherein the lysine residue is used to effect branching). A preferred branched water-soluble polymer comprises the following structure: 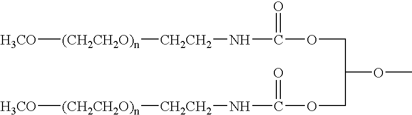
wherein each (n) is independently an integer having a value of from 3 to 4000.

Compositions

The conjugates are typically part of a composition. Generally, the composition comprises a plurality of conjugates, preferably although not necessarily, each having one, two, three or four water-soluble polymers separately covalently attached (either directly or through a spacer moiety) to one GM-CSF moiety. The compositions, however, can also comprise other conjugates having four, five, six, seven, eight or more polymers attached to any given GM-CSF moiety.

With respect to the conjugates in the composition, the composition will typically satisfy one or more of the following characteristics: at least about 85% of the conjugates in the composition will have from one to five polymers attached to the GM-CSF moiety; at least about 85% of the conjugates in the composition will have from one to four polymers attached to the GM-CSF moiety; at least about 85% of the conjugates in the composition will have from one to three polymers attached to the GM-CSF moiety; at least about 85% of the conjugates in the composition will have from one to two polymers attached to the GM-CSF moiety; at least about 85% of the conjugates in the composition will have one polymer attached to the GM-CSF moiety (i.e., be monoPEGylated); at least about 95% of the conjugates in the composition will have from one to five polymers attached to the GM-CSF moiety; at least about 95% of the conjugates in the composition will have from one to four polymers attached to the GM-CSF moiety; at least about 95% of the conjugates in the composition will have from one to three polymers attached to the GM-CSF moiety; at least about 95% of the conjugates in the composition will have from one to two polymers attached to the GM-CSF moiety; at least about 95% of the conjugates in the composition will have one polymer attached to the GM-CSF moiety (i.e., be monoPEGylated); at least about 99% of the conjugates in the composition will have from one to five polymers attached to the GM-CSF moiety; at least about 99% of the conjugates in the composition will have from one to four polymers attached to the GM-CSF moiety; at least about 99% of the conjugates in the composition will have from one to three polymers attached to the GM-CSF moiety; at least about 99% of the conjugates in the composition will have from one to two polymers attached to the GM-CSF moiety; and at least about 99% of the conjugates in the composition will have one polymer attached to the GM-CSF moiety (i.e., be monoPEGylated).

In one or more embodiments, it is preferred that the conjugate-containing composition is free or substantially free of albumin. It is also preferred that the composition is free or substantially free of proteins that do not have GM-CSF activity. Thus, it is preferred that the composition is 85%, more preferably 95%, and most preferably 99% free of albumin. Additionally, it is preferred that the composition is 85%, more preferably 95%, and most preferably 99% free of any protein that does not have GM-CSF activity. To the extent that albumin is present in the composition, exemplary compositions of the invention are substantially free of conjugates comprising a poly(ethylene glycol) polymer linking a residue of a GM-CSF moiety to albumin.

In one or more embodiments of the invention, a pharmaceutical composition is provided comprising
(i) a conjugate comprising a human GM-CSF covalently attached, either directly or through a spacer moiety comprised of one or more atoms, to a water-soluble polymer, wherein the water-soluble polymer has a weight-average molecular weight of greater than 5,000 Daltons; and
(ii) a pharmaceutically acceptable excipient,
wherein at least about 85% of the conjugates in the composition will have from one to two polymers attached to the human GM-CSF. In some instances, the pharmaceutical composition includes the proviso that when the water-soluble polymer is a branched water-soluble polymer, the branched water-soluble polymer lacks a lysine residue in which the polymeric portions are connected to amine groups of the lysine via a “—OCH₂CONHCH₂CO—” group. In still other instances, the pharmaceutical composition includes the proviso that the water-soluble polymer lacks a lysine residue (wherein the lysine residue is used to effect branching).

Control of the desired number of polymers for any given moiety can be achieved by selecting the proper polymeric reagent, the ratio of polymeric reagent to the GM-CSF moiety, temperature, pH conditions, and other aspects of the conjugation reaction. In addition, reduction or elimination of the undesired conjugates (e.g., those conjugates having four or more attached polymers) can be achieved through purification means.

For example, the water-soluble polymer-(GM-CSF) moiety conjugates can be purified to obtain/isolate different conjugated species. Specifically, the product mixture can be purified to obtain an average of anywhere from one, two, three, four, five or more PEGs per GM-CSF moiety, typically one, two or three PEGs per GM-CSF moiety. The strategy for purification of the final conjugate reaction mixture will depend upon a number of factors, including, for example, the molecular weight of the polymeric reagent employed, the particular GM-CSF moiety, the desired dosing regimen, and the residual activity and in vivo properties of the individual conjugate(s).

If desired, conjugates having different molecular weights can be isolated using gel filtration chromatography and/or ion exchange chromatography. That is to say, gel filtration chromatography is used to fractionate differently numbered polymer-to-(GM-CSF) moiety ratios [e.g., 1-mer, 2-mer, 3-mer, and so forth, wherein “1-mer” indicates 1 polymer attached to a GM-CSF moiety (or monoPEGylated when the polymer is PEG), “2-mer” indicates two polymers attached to GM-CSF moiety (or diPEGylated when the polymer is PEG), and so on] on the basis of their differing molecular weights (where the difference corresponds essentially to the average molecular weight of the water-soluble polymer portion). For example, in an exemplary reaction where a 20,000 Dalton protein is randomly conjugated to a PEG reagent having a molecular weight of about 20,000 Daltons, the resulting reaction mixture may contain unmodified protein having a molecular weight of about 20,000 Daltons, monoPEGylated protein (or “1-mer”) having a molecular weight of about 40,000 Daltons, diPEGylated protein (or 2-mer”) having a molecular weight of about 60,000 Daltons, and so forth.

While this approach can be used to separate PEG and other water-soluble polymer-(GM-CSF) moiety conjugates having different molecular weights, this approach is generally ineffective for separating positional isomers having different polymer attachment sites within the GM-CSF moiety. For example, gel filtration chromatography can be used to separate from each other mixtures of 1-mers, 2-mers, 3-mers, and so forth, although each of the recovered PEG-mer compositions may contain PEGs attached to different reactive amino groups (e.g., lysine residues) within GM-CSF moiety.

Gel filtration columns suitable for carrying out this type of separation include Superdex™ and Sephadex™ columns available from Amersham Biosciences (Piscataway, N.J.). Selection of a particular column will depend upon the desired fractionation range desired. Elution is generally carried out using a suitable buffer, such as phosphate, acetate, or the like. The collected fractions may be analyzed by a number of different methods, for example, (i) absorbance at 280 nm for protein content, (ii) dye-based protein analysis using bovine serum albumin as a standard, (iii) iodine testing for PEG content (Sims et al. (1980) Anal. Biochem, 107:60-63), (iv) sodium dodecyl sulfate polyacrylamide gel electrophoresis (SDS PAGE), followed by staining with barium iodide, and higher performance liquid chromatography.

Separation of positional isomers can be carried out by reverse phase chromatography using reverse phase-high performance liquid chromatography (RP-HPLC) methods using, for example, a C18 column or C3 column (Amersham Biosciences or Vydac) or by ion exchange chromatography using an ion exchange column, e.g., a Sepharose™ ion exchange column available from Amersham Biosciences. Either approach can be used to separate polymer-active agent isomers having the same molecular weight (positional isomers).

The compositions are preferably substantially free of proteins that do not have GM-CSF activity. In addition, the compositions preferably are substantially free of all other noncovalently attached water-soluble polymers. In some circumstances, however, the composition can contain a mixture of water-soluble polymer-(GM-CSF) moiety conjugates and unconjugated GM-CSF.

Optionally, the composition of the invention further comprises a pharmaceutically acceptable excipient. If desired, the pharmaceutically acceptable excipient can be added to a conjugate to form a composition.

Exemplary pharmaceutically acceptable excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The composition can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the conjugate (i.e., the conjugate formed between the active agent and the polymeric reagent) in the composition will vary depending on a number of factors, but will optimally be a therapeutically effective amount when the composition is stored in a unit dose container (e.g., a vial). In addition, the pharmaceutical preparation can be housed in a syringe. A therapeutically effective amount can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient, with concentrations less than 30% by weight most preferred.

These foregoing pharmaceutically acceptable excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

A method for making a conjugate is also provided, the method comprising contacting, under conjugation conditions, a GM-CSF moiety with a polymeric reagent. As provided herein, the method does not necessarily involve carrying out protecting and deprotecting steps. The Experimental section below provides exemplary approaches for making conjugates. Once a conjugate is prepared, a pharmaceutically acceptable excipient can be added to the conjugate to provide a pharmaceutical composition.

The pharmaceutical compositions encompass all types of formulations and in particular those that are suited for injection, e.g., powders or lyophilates that can be reconstituted as well as liquids. Examples of suitable diluents for reconstituting solid compositions prior to injection include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

In one or more embodiments of the invention, a method is provided, the method comprising delivering a conjugate to a patient, the method comprising the step of administering to the patient a pharmaceutical composition as provided herein. This method has utility as, among other things, a method for screening the pharmaceutical composition for toxicity (either of itself against a known standard or of other compositions to test relatively toxicities). In addition the method may be used to treat a patient suffering from a condition that is responsive to treatment with conjugate by administering a therapeutically effective amount of the pharmaceutical composition. Administration can be effected by, for example, intravenous injection, intramuscular injection, subcutaneous injection, and so forth. Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others.

As previously stated, the method of delivering may be used to treat a patient having a condition that can be remedied or prevented by administration of the conjugate. Those of ordinary skill in the art appreciate which conditions a specific conjugate can effectively treat. For example, the conjugate can be administered to the patient prior to, simultaneously with, or after administration of a chemotherapy agent. In addition, the conjugate can be administered to a patient undergoing bone marrow transplantation (such as a patient suffering from acute myelogenous leukemia), wherein administration occurs prior to, simultaneously with, or after the bone marrow transplant (either autologous or allogenic). Furthermore, the conjugate can be used in the treatment of cancers via enhancement of the cytotoxic activity of peripheral monocytes and lymphocytes, mucositis, stomatitis, diarrhea, wound healing, pulmonary alveolar proteinosis, and hypercholesterolemia. Finally, the conjugates can also be used as a vaccine adjuvant.

The actual dose to be administered will vary depending upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, on a weight basis, a therapeutically effective amount will range from about 0.001 mg to 100 mg, preferably in doses from 0.01 mg/day to 75 mg/day, and more preferably in doses from 0.10 mg/day to 50 mg/day. On an activity basis, corresponding doses based on international units of activity can be calculated by one of ordinary skill in the art.

The unit dosage of any given conjugate (again, preferably provided as part of a pharmaceutical composition) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All articles, books, patents and other publications referenced herein are hereby incorporated by reference in their entireties.

### Experimental

The practice of the invention will employ, unless otherwise indicated, conventional techniques of organic synthesis and the like, which are within the skill of the art. Such techniques are fully explained in the literature. Reagents and materials are commercially available unless specifically stated to the contrary. See, for example, J. March, Advanced Organic Chemistry: Reactions Mechanisms and Structure, 4th Ed. (New York: Wiley-Interscience, 1992), supra.

In the following examples, efforts have been made to ensure accuracy with respect to numbers used (e.g., amounts, temperatures, etc.) but some experimental error and deviation should be accounted for. Unless indicated otherwise, temperature is in degrees C and pressure is at or near atmospheric pressure at sea level.

Although other abbreviations known by one having ordinary skill in the art will be referenced, other reagents and materials will be used, and other methods known by one having ordinary skill in the art will be used, the following list and methods description is provided for the sake of convenience.

Abbreviations:

NaCNBH₃ sodium cyanoborohydride

HCl hydrochloric acid

HEPES 4-(2-hydroxyethyl)-1-piperazineethanesulfonic acid

K or kDa kiloDaltons

SEC Size exclusion chromatography

HPLC high performance liquid chromatography

SDS-PAGE sodium dodecyl sulfate-polyacrylamide gel electrophoresis

SDS-PAGE Analysis

Samples indicated to be were analyzed by sodium dodecyl sulfate-polyacrylamide gel electrophoresis (SDS-PAGE) using a Bio-Rad system (Mini-PROTEAN In Precast Gel Electrophoresis System). Samples were mixed with sample buffer. Then, the prepared samples were loaded onto a gel and run for approximately thirty minutes.

SEC-HPLC Analysis

Size exclusion chromatography (SEC-HPLC) analysis was performed on an Agilent 1100 HPLC system (Agilent). Samples were analyzed using a Shodex protein KW-804 column (300×8 mm, Phenomenex), at pH 7.4. The flow rate for the column was 0.5 mulminute. Eluted protein and PEG-protein conjugates were detected using UV at 280 nm.

Anion-Exchange Chromatography

A HiTrap Q Sepharose HP anion exchange column (Amersham Biosciences) was used with the AKTAprime system (Amersham Biosciences) to purify the PEGylated GM-CSF conjugates prepared in Example 1 through 6. For each conjugate solution prepared, the conjugate solution was loaded on a column that was pre-equilibrated in 20 mM Tris buffer, pH 7.5 (buffer A) and then washed with ten column volumes of buffer A to remove any unreacted PEG reagent. Subsequently, a gradient of buffer A with 0-100% buffer B (20 mM Tris with 0.5 M NaCl buffer, pH 7.5) was raised. The eluent was monitored by UV detector at 280 nm. Any higher-mers (e.g., 3-mers, 4-mers, and so forth) eluted first, next followed by 2-mers, and then 1-mers, and finally unconjugated GM-CSF. The fractions were pooled according to the chromatogram, and the purity of the individual conjugate was determined by SEC-HPLC or SDS-PAGE.

Recombinant human GM-CSF (hGM-CSF) corresponding to the amino acid sequence of SEQ ID NO: 2. was used in Examples 1-6 and was obtained from a commercial source. A stock hGM-CSF solution was prepared by ensuring that the recombinant human GM-CSF existed in an amine-free buffer, using (if necessary) a buffer exchange technique known to those of ordinary skill in the art.

### EXAMPLE 1

### PEGylation of hGM-CSF with Branched mPEG-N-Hydroxysuccinimide Derivative, 40 kDa

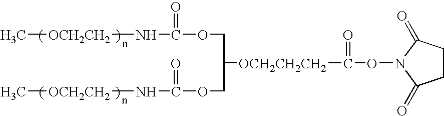 Branched mPEG-N-Hydroxysuccinimide Derivative, 40 kDa, (“mPEG2-NHS”)

mPEG2-NHS, 40 kDa, stored at −20° C. under argon, was warmed to ambient temperature. A five-fold excess (relative to the amount of hGM-CSF in a measured aliquot of the stock hGM-CSF solution) of the warmed mPEG2-NHS was dissolved in 2mM HCl to form a 10% reagent solution. The 10% reagent solution was quickly added to the aliquot of stock hGM-CSF solution (1 mg/mL in sodium phosphate buffer, pH 7.0) and mixed well. After the addition of the PEG reagent, the pH of the reaction mixture was determined and adjusted to 7.0 using conventional techniques. To allow for coupling of the mPEG2-NHS to hGM-CSF via an amide linkage, the reaction solution was placed on a Slow Speed Lab Rotator overnight to facilitate conjugation at room temperature. The reaction was quenched with Tris buffer. The conjugate solution was characterized as provided below.

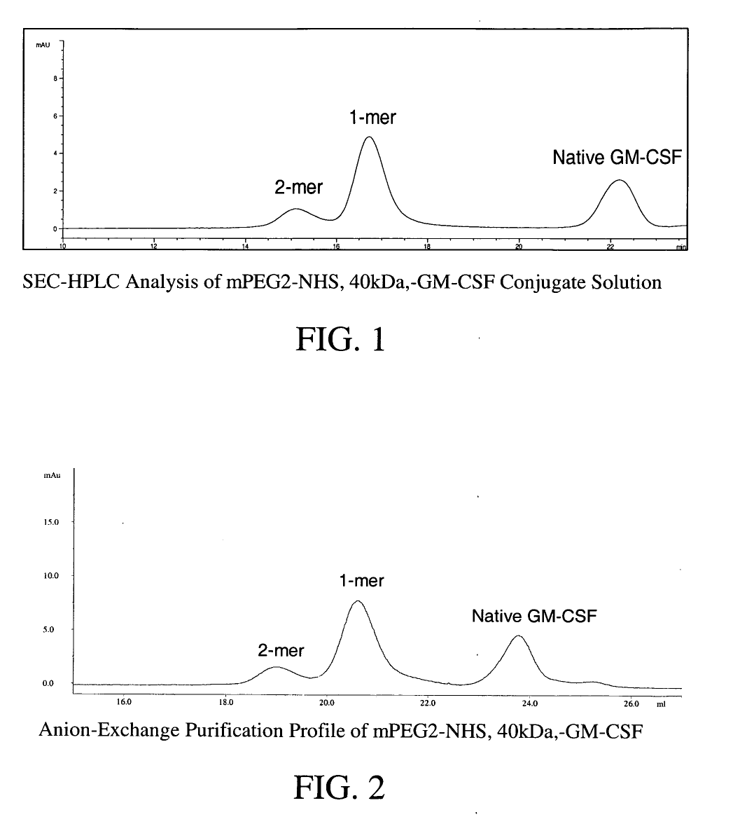

FIG. 1 shows the chromatogram following the SEC-HPLC analysis of the conjugate solution. The PEGylation reaction yielded 57% 1-mer (mono-conjugate or one PEG attached to hGM-CSF) and 13% 2-mer (di-conjugate or two PEGs attached to hGM-CSF) species.

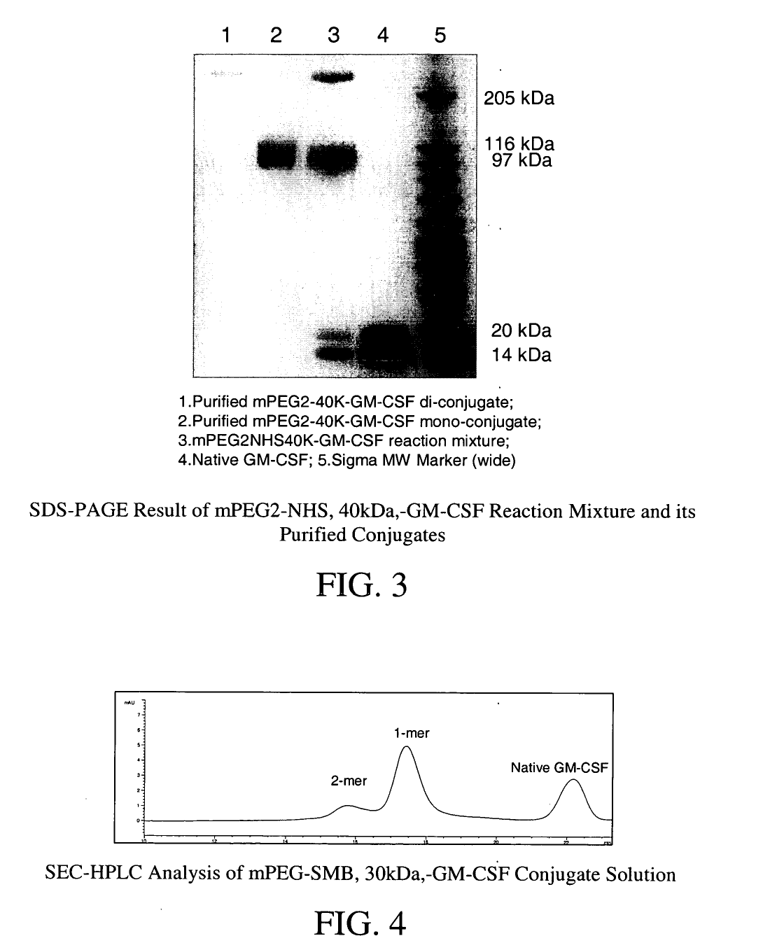

Anion-exchange chromatography was used to purify the conjugates. FIG. 2 shows the chromatogram following anion-exchange purification. The conjugate fractions were collected and analyzed by SDS-PAGE (FIG. 3). The purified conjugates were up to 100% pure.

Using this same approach, other conjugates can be prepared using mPEG2-NHS having other weight average molecular weights.

### EXAMPLE 2

### PEGylation of hGM-CSF with Linear mPEG-Succinimidyl

### α-Methylbutanoate Derivative, 30 kDa

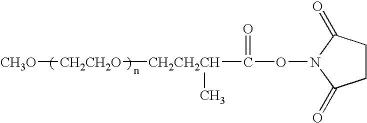 Linear mPEG-Succinimidyl α-Methylbutanoate Derivative, 30 kDa (“mPEG-SMB”)

mPEG-SMB, 30 kDa, stored at −20° C. under argon, was warmed to ambient temperature. A ten-fold excess (relative to the amount of hGM-CSF in a measured aliquot of the stock hGM-CSF solution) of the warmed MPEG-SMB was dissolved in 2 mM HCl to form a 10% reagent solution. The 10% reagent solution was quickly added to the aliquot of stock hGM-CSF solution (1 mg/mL in sodium phosphate buffer, pH 7.0) and mixed well. After the addition of the mPEG-SMB, the pH of the reaction mixture was determined and adjusted to 7.0 using conventional techniques. To allow for coupling of the mPEG-SMB to hGM-CSF via an amide linkage, the reaction solution was placed on a Slow Speed Lab Rotator overnight to facilitate conjugation at room temperature. The reaction was quenched with Tris buffer. The conjugate solution was characterized as provided below.

FIG. 4 shows the SEC-HPLC chromatogram of the conjugate solution. The SEC-HPLC analysis reveals the PEGylation reaction yielded 58% 1-mer (mono-conjugate or one PEG attached to hGM-CSF) and 14% 2-mer (di-conjugate or two PEGs attached to hGM-CSF) species. An anion-exchange chromatography method using Q Sepharose High Performance and Tris buffer was also used to purify the conjugates. The separation profile of the conjugate species was similar to that shown in FIG. 2.

Using this same approach, other conjugates can be prepared using mPEG-SMB having other weight average molecular weights.

### EXAMPLE 3

### PEGylation of hGM-CSF with mPEG-Piperidone, 20 kDa

mPEG-Piperidone (mPEG-PIP) having a molecular weight of 20,000 Daltons is obtained from Nektar Therapeutics (Huntsville, Ala.). The basic structure of the polymeric reagent is provided below: 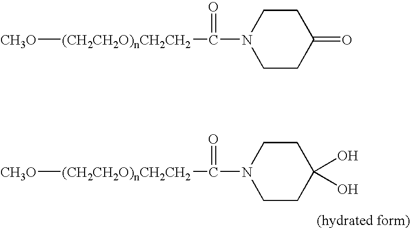
Linear mPEG-Piperidone Derivative, 20 kDa (“mPEG-PIP”)

mPEG-PIP, 20 kDa, stored at −20° C. under argon, was warmed to ambient temperature. A fifty to one hundred-fold excess (relative to the amount of hGM-CSF in a measured aliquot of the stock hGM-CSF) of the warmed MPEG-PIP was dissolved in 10 mM sodium phosphate (pH 7.0) to form a 10% reagent solution. The 10% reagent solution was quickly added to the aliquot of stock hGM-CSF solution (1 mg/mL in sodium phosphate buffer, pH 7.0) and mixed well. After the addition of the MPEG-PIP, the pH of the reaction mixture was determined and adjusted to 7.0 using conventional techniques, followed by mixing for thirty minutes. A reducing agent, sodium cyanoborohydride, was then added to make 13 mM NaCNBH₃. The reaction solution was placed on a Slow Speed Lab Rotator overnight to facilitate conjugation at room temperature. The reaction was quenched with Tris buffer. The conjugate solution was characterized as provided below.

The PEGylation reaction yielded over 30% of 1-mer (mono-conjugate or one PEG attached to hGM-CSF). Due to the high selectivity of the PEG ring structure, little 2-mer was resulted.

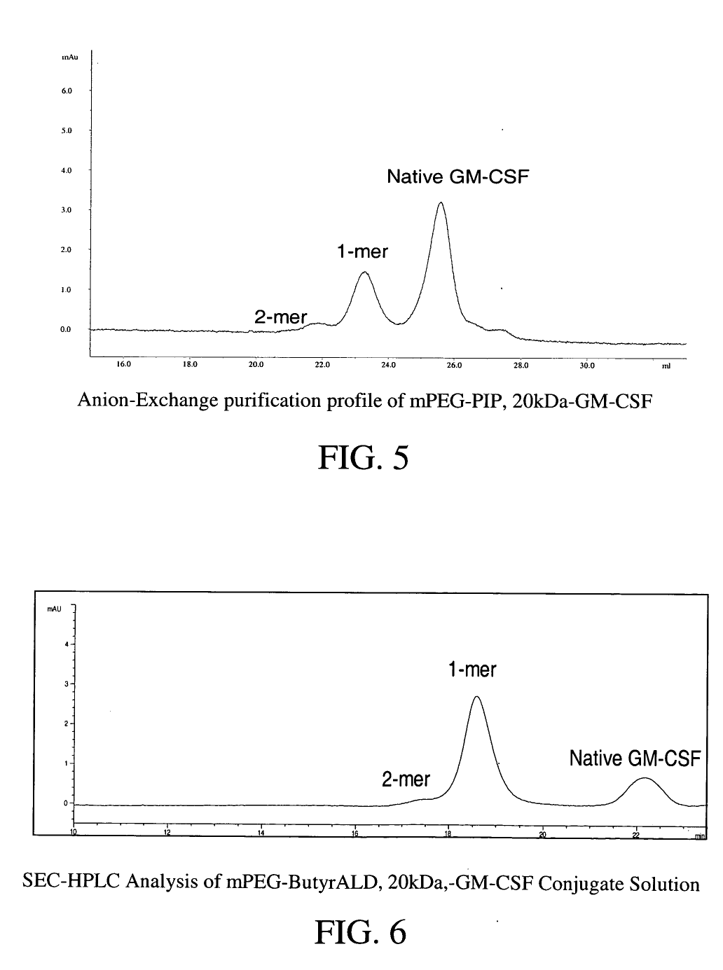

An anion-exchange chromatography method was also used to purify the conjugates. FIG. 5 depicts the anion-exchange purification profile.

### EXAMPLE 4

### PEGylation of hGM-CSF with Linear mPEG-Butyraldehyde Derivative, 20 kDa

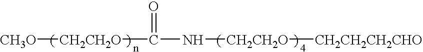 Linear mPEG-Butyraldehyde Derivative, 20 kDa (“mPEG-ButyrALD”)

mPEG-ButyrALD, 20 kDa, stored at −20° C. under argon, was warmed to ambient temperature. A thirty-fold excess (relative to the amount of hGM-CSF in a measured aliquot of the stock hGM-CSF) of the warmed mPEG-ButryALD was dissolved in Milli-Q H₂O to form a 10% reagent solution. The 10% reagent solution was quickly added to the aliquot of stock hGM-CSF solution (1 mg/mL in sodium phosphate buffer, pH 7.0) and mixed well. After the addition of the mPEG-ButryALD, the pH of the reaction mixture was determined and adjusted to 6.0 using conventional techniques, followed by mixing for thirty minutes. A reducing agent, sodium cyanoborohydride was then added to make 9 mM NaCNBH₃. The reaction solution was placed on a Slow Speed Lab Rotator overnight to facilitate conjugation at room temperature. The reaction was quenched with Tris buffer. The conjugate solution was characterized as provided below.

The aldehyde group of mPEG-ButyrALD can react with the primary amines associated with hGM-CSF and covalently bond to them via secondary amine upon reduction by a reducing reagent such as sodium cyanoborohydride. Because the PEGylation reaction was carried at pH 6.0, attachment of the PEG derivative to hGM-CSF was more selective to the N-terminal. FIG. 6 shows the SEC-HPLC chromatogram of the conjugate solution. The PEGylation reaction yielded 75% 1-mer (one PEG attached to hGM-CSF or monoPEGylated) and 4% 2-mer (di-conjugate or two PEGs attached to hGM-CSF) species. An anion-exchange chromatography method using Q Sepharose High Performance and Tris buffer was also used to purify the conjugates. The separation profile of the conjugate species was similar to that shown in FIG. 5.

Using this same approach, other conjugates can be prepared using mPEG-ButyrALD having other weight average molecular weights.

### EXAMPLE 5

### PEGylation of GM-CSF with Linear mPEG-Butyraldehyde Derivative, 30 kDa

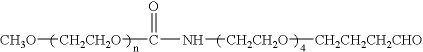 Linear mPEG-Butyraldehyde Derivative, 30 kDa (“mPEG-ButyrALD”)

mPEG-ButyrALD, 30 kDa, stored at −20° C. under argon, was warmed to ambient temperature. A thirty-fold excess (relative to the amount of hGM-CSF in a measured aliquot of the stock hGM-CSF) of the warmed mPEG-ButryALD was dissolved in Milli-Q H₂O to form a 10% reagent solution. The 10% reagent solution was quickly added to the aliquot of stock hGM-CSF solution (1 mg/mL in sodium phosphate buffer, pH 7.0) and mixed well. After the addition of the mPEG-ButryALD, the pH of the reaction mixture was determined and adjusted to 6.0 using conventional techniques, followed by mixing for thirty minutes. A reducing agent, sodium cyanoborohydride was then added to make 9 mM NaCNBH₃ The reaction solution was placed on a Slow Speed Lab Rotator overnight to facilitate conjugation at room temperature. The reaction was quenched with Tris buffer. The conjugate solution was characterized as provided below.

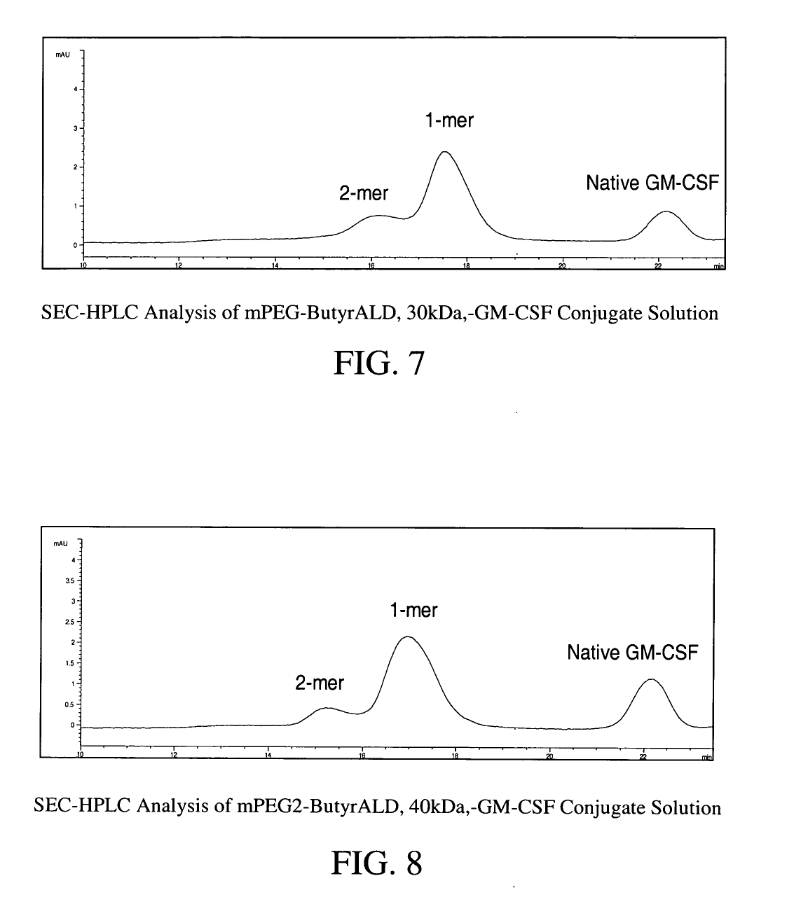

The aldehyde group of mPEG-ButyrALD can react with the primary amines associated with hGM-CSF and covalently bond to them via secondary amine upon reduction by a reducing reagent such as sodium cyanoborohydride. Because the PEGylation reaction was carried at pH 6.0, attachment of the PEG derivative to hGM-CSF was more selective to the N-terminal. FIG. 7 shows the SEC-HPLC chromatogram of the conjugate solution. The PEGylation reaction yielded 63% 1-mer (one PEG attached to hGM-CSF or monoPEGylated) and 20% 2-mer (di-conjugate or two PEGs attached to hGM-CSF) species. An anion-exchange chromatography method using Q Sepharose High Performance and Tris buffer was also used to purify the conjugates. The separation profile of the conjugate species was similar to that shown in FIG. 2.

Using this same approach, other conjugates can be prepared using mPEG-ButyrALD having other weight average molecular weights.

### EXAMPLE 6

### PEGylation of hGM-CSF with Branched mPEG-Butyraldehyde Derivative, 40 kDa

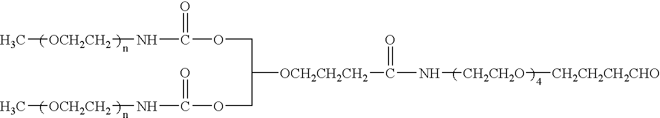 Branched mPEG-Butyraldehyde Derivative, 40 kDa (“mPEG2-ButyrALD”)

mPEG2-ButyrALD, 40 kDa, stored at −20° C. under argon, was warmed to ambient temperature. A thirty-fold excess (relative to the amount of hGM-CSF in a measured aliquot of the stock hGM-CSF) of the warmed mPEG2-ButryALD was dissolved in Milli-Q H₂O to form a 10% reagent solution. The 10% reagent solution was quickly added to the aliquot of stock hGM-CSF solution (1 mg/mL in sodium phosphate buffer, pH 7.0) and mixed well. After the addition of the mPEG2-ButryALD, the pH of the reaction mixture was determined and adjusted to 6.0 using conventional techniques, followed by mixing for thirty minutes. A reducing agent, sodium cyanoborohydride was then added to make 9 mM NaCNBH₃. The reaction solution was placed on a Slow Speed Lab Rotator overnight to facilitate conjugation at room temperature. The reaction was quenched with Tris buffer. The conjugate solution was characterized as provided below.

The aldehyde group of mPEG2-ButyrALD can react with the primary amines associated with hGM-CSF and covalently bond to them via secondary amine upon reduction by a reducing reagent such as sodium cyanoborohydride. Because the PEGylation reaction was carried at pH 6.0, attachment of the PEG derivative to hGM-CSF was more selective to the N-terminal. FIG. 8 shows the SEC-HPLC chromatogram of the conjugate solution. The PEGylation reaction yielded 65% 1-mer (one PEG attached to hGM-CSF or monoPEGylated) and 10% 2-mer (di-conjugate or two PEGs attached to hGM-CSF) species. An anion-exchange chromatography method using Q Sepharose High Performance and Tris buffer was also used to purify the conjugates. The separation profile of the conjugate species was similar to that shown in FIG. 2.

Using this same approach, other conjugates can be prepared using mPEG2-ButyrALD having other weight average molecular weights.

### EXAMPLE 7

### PEGylation of GM-CSF with mPEG-SBA

mPEG-Succinimidyl butanoate having a molecular weight of 20,000 Daltons is obtained from Nektar Therapeutics, (Huntsville, Ala.). The basic structure of the polymer reagent is provided below:

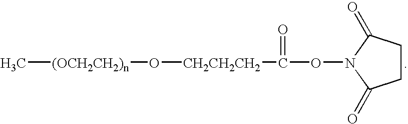

GM-CSF is dissolved in deionized water, to which is added triethylamine to raise the pH to 7.2-9. To this solution is then added a 1.5 to 10-fold molar excess of mPEG-SBA. The resulting mixture is stirred at room temperature for several hours.

The reaction mixture is analyzed by SDS-PAGE to determine the degree of PEGylation of the protein.

### EXAMPLE 8

### Conjugation of Cysteine-Inserted GM-CSF with MPEG-MAL, 20K

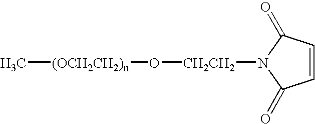 mPEG MAL, 20K

GM-CSF is inserted with one or more cysteine residues according to the process described in WO 90/12874.

MPEG-MAL, 20K, stored at −20° C. under argon, is warmed to ambient temperature. A five- to twenty-fold excess of the warmed mPEG-MAL, 20K, is dissolved in deionized water to make a 10% mPEG MAL solution. The mPEG MAL solution is quickly added to an aliquot of stock GM-CSF solution (1 mg/mL in 50 mM HEPES, pH 7.0) and is mixed well. After one hour of reaction at room temperature, the reaction vial is transferred to the cold room and the reaction is allowed to proceed overnight at 4° C. on Rotomix (slow speed, Thermolyne).

The conjugate mixture is purified using gel filtration chromatography. A size exclusion chromatography method is developed for analyzing the reaction mixtures, and the final products. SDS-PAGE analysis is also used for the characterization of the samples.

### EXAMPLE 9

### Conjugation of GM-CSF with MPEG-MAL, 30K

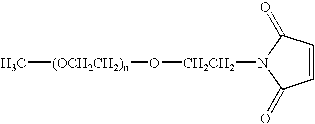 mPEG MAL, 30K

GM-CSF is inserted with one or more cysteine residues according to the process described in WO 90/12874.

mPEG-MAL, 30K, stored at −20° C. under argon, is warmed to ambient temperature. A five- to twenty-fold excess of the warmed mPEG-MAL, 30K, is dissolved in deionized water to make a 10% mPEG MAL solution. The mPEG MAL solution is quickly added to an aliquot of stock GM-CSF solution (1 mg/mL in 50 mM HEPES, pH 7.0) and is mixed well. After one hour of reaction at room temperature, the reaction vial is transferred to the cold room and the reaction is allowed to proceed overnight at 4° C. on Rotomix (slow speed, Thermolyne).

The conjugate mixture is purified using gel filtration chromatography. A size exclusion chromatography method is developed for analyzing the reaction mixtures, and the final products. SDS-PAGE analysis is also used for the characterization of the samples.

### EXAMPLE 10

### In-vitro Activity of Exemplary (GM-CSF)-PEG Conjugates

The in-vitro activities of the conjugates described in Examples 1-6 are determined. All of the conjugates tested are bioactive.

### EXAMPLES 11-19

Each of Examples 1-9 is replicated except that the GM-CSF moiety of SEQ ID NO: 2 is replaced with SEQ ID NO: 1 GM-CSF.

### EXAMPLE 20

### In-vitro Activity Conjugates

The in-vitro activities of the conjugates described in Examples 11-19 are determined. All of the conjugates tested are bioactive.

What is claimed is:

1. A conjugate comprising the following structure: 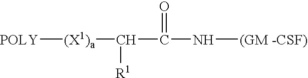
wherein:
POLY is a water-soluble polymer;
(a) is either zero or one;
X¹, when present, is a spacer moiety comprised of one or more atoms;
R¹ is an organic radical;
GM-CSF is a GM-CSF moiety.

2. The conjugate of claim 1, comprising the following structure: 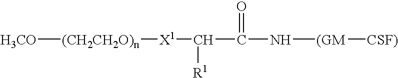
wherein:
(n) is an integer having a value of from 3 to 4000;
X¹ is as previously defined;
R¹ is selected from the group consisting of methyl, ethyl, propyl, and isopropyl; and
GM-CSF is a human GM-CSF.

3. The conjugate of claim 1, comprising the following structure: 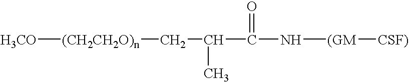
wherein (n) an integer having a value of from 3 to 4000 and GM-CSF is a human GM-CSF.

4. A conjugate comprising the following structure: 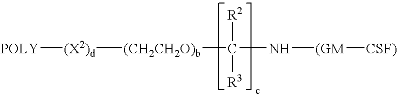
wherein:
POLY is a water-soluble polymer;
(d) is either zero or one;
X², when present, is a spacer moiety comprised of one or more atoms;
(b) is an integer having a value of one through ten;
(c) is an integer having a value of one through ten;
R², in each occurrence, is independently H or an organic radical;
R³, in each occurrence, is independently H or an organic radical; and
GM-CSF is a GM-CSF moiety.

5. The conjugate of claim 4, comprising the following structure: 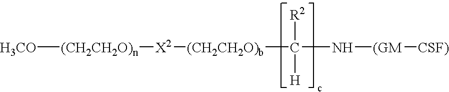
wherein:
(n) is an integer having a value of from 3 to 4000;
X² is as previously defined;
(b) is 2 through 6;
(c) is 2 through 6;
R², in each occurrence, is independently H or lower alkyl; and
GM-CSF is a human GM-CSF.

6. The conjugate of claim 5, comprising the following structure: 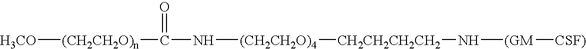
wherein (n) is an integer having a value of from 3 to 4000, and GM-CSF is a human GM-CSF.

7. The conjugate of claim 4, comprising the following structure: 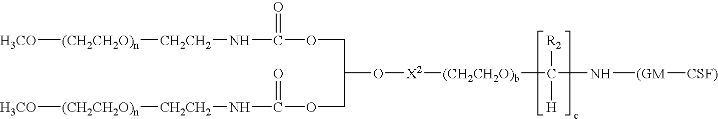
wherein:
each (n) is independently an integer having a value of from 3 to 4000;
X is as previously defined;
(b) is 2 through 6;
(c) is 2 through 6;
R², in each occurrence, is independently H or lower alkyl; and
GM-CSF is a GM-CSF moiety.

8. The conjugate of claim 5, comprising the following structure: 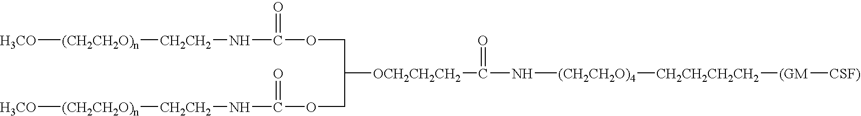
wherein:
each (n) is independently an integer having a value of from 3 to 4000; and
GM-CSF is a human GM-CSF.

9. A conjugate comprising a GM-CSF moiety comprising an internal amine covalently attached, either directly or through a spacer moiety comprised of one or more atoms, to a branched water-soluble polymer.

10. The conjugate of claim 9, wherein the conjugate further comprises an additional branched water-soluble polymer covalently attached, either directly or through a spacer moiety comprised of one or more atoms, to the N-terminal amino acid residue.

11. The conjugate of claim 9, wherein the branched water-soluble polymer lacks a lysine residue in which polymers are connected to amine groups of the lysine residue via a —OCH₂CONHCH₂CO— linkage.

12. The conjugate of claim 9, wherein the branched water-soluble polymer comprises the following structure: 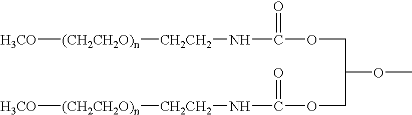
wherein each (n) is independently an integer having a value of from 3 to 4000.

13. The conjugate of claim 1, 4 or 9, wherein the water-soluble polymer in each conjugate is selected from the group consisting of a poly(alkylene oxide), poly(vinyl pyrrolidone), poly(vinyl alcohol), polyoxazoline, and poly(acryloylmorpholine).

14. The conjugate of claim 13, wherein each water-soluble polymer is a poly(alkylene oxide).

15. The conjugate of claim 14, wherein each poly(alkylene oxide) is a poly(ethylene glycol).

16. The conjugate of claim 15, wherein the poly(ethylene glycol) is terminally capped with an end-capping moiety selected from the group consisting hydroxy, alkoxy, substituted alkoxy, alkenoxy, substituted alkenoxy, alkynoxy, substituted alkynoxy, aryloxy and substituted aryloxy.

17. The conjugate of claim 15, wherein the poly(ethylene glycol) is terminally capped with methoxy.

18. The conjugate of claim 1, 4 or 9, wherein the water-soluble polymer has a total weight-average molecular weight in the range of from greater than 5,000 Daltons to about 150,000 Daltons.

19. The conjugate of claim 18, wherein the poly(ethylene glycol) has a total weight-average molecular weight in the range of from about 6,000 Daltons to about 100,000 Daltons.

20. The conjugate of claim 19, wherein the poly(ethylene glycol) has a total weight-average molecular weight in the range of from about 15,000 Daltons to about 85,000 Daltons.

21. The conjugate of claim 20, wherein the poly(ethylene glycol) has a total weight-average molecular weight in the range of from about 20,000 Daltons to about 85,000 Daltons.

22. The conjugate of claim 21, wherein the poly(ethylene glycol has a total weight average molecular weight in the range of from about 20,000 Daltons to about 60,000 Daltons.

23. The conjugate of claim 1 or 4, wherein the water-soluble polymer is branched.

24. The conjugate of claim 1, 4 or 9, wherein the GM-CSF moiety is selected from the group consisting of a human GM-CSF, and biologically active fragments, deletion variants, substitution variants or addition variants of any of the foregoing.

25. The conjugate of claim 1, 4 or 9, wherein the GM-CSF moiety is recombinantly derived.

26. The conjugate of claim 1, 4 or 9, wherein not more than three water-soluble polymers are attached to the GM-CSF moiety.

27. The conjugate of claim 1, 4 or 9 in diPEGylated form.

28. The conjugate of claim 1, 4 or 9 in monoPEGylated form.

29. The conjugate of claim 1, 4 or 9, wherein the GM-CSF moiety is nonglycosylated.

30. The conjugate of claim 1, 4 or 9, wherein the GM-CSF moiety is glycosylated.

31. A pharmaceutical composition comprising:
(i) a conjugate comprising a human GM-CSF covalently attached, either directly or through a spacer moiety comprised of one or more atoms, to a water-soluble polymer, wherein the water-soluble polymer has a weight-average molecular weight of greater than 5,000 Daltons; and
(ii) a pharmaceutically acceptable excipient,
wherein at least about 85% of the conjugates in the composition have from one to two polymers attached to the human GM-CSF.

32. The composition of claim 31, wherein each water-soluble polymer in each conjugate is selected from the group consisting of a poly(alkylene oxide), poly(vinyl pyrrolidone), poly(vinyl alcohol), polyoxazoline, and poly(acryloylmorpholine).

33. The composition of claim 32, wherein each water-soluble polymer is a poly(alkylene oxide).

34. The composition of claim 33, wherein each poly(alkylene oxide) is a poly(ethylene glycol).

35. The composition of claim 34, wherein the poly(ethylene glycol) is terminally capped with an end-capping moiety selected from the group consisting hydroxy, alkoxy, substituted alkoxy, alkenoxy, substituted alkenoxy, alkynoxy, substituted alkynoxy, aryloxy and substituted aryloxy.

36. The composition of claim 34, wherein the poly(ethylene glycol) is terminally capped with methoxy.

37. The composition of claim 34, wherein the poly(ethylene glycol) has a total weight-average molecular weight in the range of from greater than 5,000 Daltons to about 150,000 Daltons.

38. The composition of claim 37, wherein the poly(ethylene glycol) has a total weight-average molecular weight in the range of from about 6,000 Daltons to about 100,000 Daltons.

39. The composition of claim 38, wherein the poly(ethylene glycol) has a total weight-average molecular weight in the range of from about 15,000 Daltons to about 85,000 Daltons.

40. The composition of claim 39, wherein the poly(ethylene glycol) has a total weight-average molecular weight in the range of from about 20,000 Daltons to about 85,000 Daltons.

41. The composition of claim 40, wherein the poly(ethylene glycol) has a total weight average molecular weight in the range of from about 20,000 Daltons to about 60,000 Daltons.

42. The composition of claim 41, wherein each water-soluble polymer in each conjugate is a linear water-soluble polymer.

43. The composition of claim 31, wherein each water-soluble polymer in each conjugate is a branched water-soluble polymer.

44. The composition of claim 43, wherein the branched water-soluble polymer lacks a lysine residue in which polymers are connected to amine groups of the lysine residue via a —CH₂CONHCH₂CO— group.

45. The composition of claim 31, with the proviso that when the water-soluble polymer is a branched water-soluble polymer, the branched water-soluble polymer lacks a lysine residue used to effect branching.

46. The composition of claim 43, wherein the branched water-soluble polymer comprises the following structure: 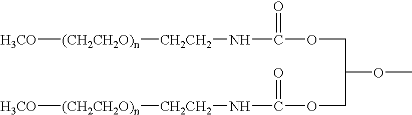
wherein each (n) is independently an integer having a value of from 3 to 4000.

47. The composition of claim 31, wherein the human GM-CSF comprises an amino acid sequence of SEQ ID NO: 1.

48. The composition of claim 31, wherein the human GM-CSF comprises an amino acid sequence of SEQ ID NO: 2.

49. The composition of claim 31, wherein the human GM-CSF is recombinantly derived.

50. The composition of claim 48, wherein the human GM-CSF is nonglycosylated.

51. The composition of claim 48, wherein the human GM-CSF is glycosylated.

52. A pharmaceutical composition comprising: (i) the conjugate of claim 1, 4 or 9; and (ii) a pharmaceutically acceptable excipient.

53. The composition of claim 31 or 52, wherein the composition is substantially free of albumin.

54. The composition of claim 31 or 52, wherein the composition is substantially free of proteins that do not have GM-CSF activity.

55. The composition of claim 31 or 52, wherein the composition is substantially free of noncovalently attached water-soluble polymers.

56. The composition of claim 31 or 52, in lyophilized form.

57. The composition of claim 31 or 52, in liquid form.

58. The composition of claim 31 or 52, wherein at least about 90% of the conjugates in the composition have from one to two polymers attached to the human GM-CSF.

59. The composition of claim 58, wherein at least about 95% of the conjugates in the composition have from one to two polymers attached to the human GM-CSF.

60. A method for delivering a conjugate to a patient, the method comprising the step of administering to the patient a pharmaceutical composition of claim 31 or 52.

61. The method of claim 60, carried out by subcutaneous injection.

62. A method for making a conjugate comprising contacting, under conjugation conditions, a GM-CSF moiety with a polymeric reagent to result in a conjugate of claim 1, 4 or 9.

63. The method of claim 62, wherein protecting and deprotecting steps are not performed.


SEQUENCE LISTING (3 sequences)

SEQ ID NO: 1 - protein, 127 aa, Homo sapiens [sequence omitted]

SEQ ID NO: 2 - protein, 127 aa, Homo sapiens
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - protein, 127 aa, Homo sapiens
APARSPSPSTQPWEHVNAIQEARLLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE